(19) United States
(12) Patent Application Publication
(10) Pub. No.: US 2015/0291565 A1
(43) Pub. Date: Oct. 15, 2015

(54) INDOLE COMPOUNDS AND THEIR USE AS ANTIMICROBIALS

(71) Applicant: Memorial Sloan-Kettering Cancer Center, New York, NY (US)

(72) Inventors: Hakim Djaballah, Scarsdale, NY (US); Michael Glickman, Pelham, NY (US)

(73) Assignee: MEMORIAL SLOAN-KETTERING CANCER CENTER, New York, NY (US)

(21) Appl. No.: 14/441,905

(22) PCT Filed: Nov. 12, 2013
(86) PCT No.: PCT/US2013/069639

§ 371 Date: May 11, 2015

Related U.S. Application Data
(60) Provisional application No. 61/725,683, filed on Nov. 13, 2012.

(51) Int. Cl. C07D 403/14; C07D 401/14; C07D 403/12; C07D 403/04

(52) U.S. Cl. CPC C07D 403/14; C07D 403/04; C07D 401/14; C07D 403/12

(57) ABSTRACT

Indole compounds of formula I, as well as compositions including the compounds and methods for their use, are disclosed that are useful for treating bacterial and/or fungal infections. Indole compounds of formula I, compositions and methods are disclosed that also are useful for killing or inhibiting the growth of bacteria and/or fungus:

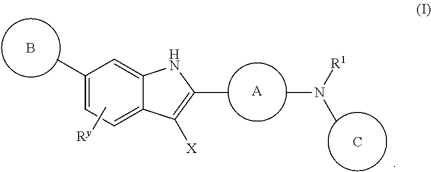

### CROSS REFERENCE TO RELATED APPLICATIONS

This application claims priority from U.S. Provisional Application No. 61/725,683, filed Nov. 13, 2012. This Provisional Application is hereby incorporated by reference in its entirety herein.

### GOVERNMENT RIGHTS STATEMENT

This invention was partially supported with U.S. Government under Cancer Center Support Grant Number 5 P30 CA008748-47 awarded by NIH/NCI. The U.S. Government has certain rights in the invention.

### TECHNICAL FIELD

The invention relates to indole-containing chemical compounds having antibacterial and antifungal activity.

### BACKGROUND INFORMATION

There are many bacteria and fungi that cause infections in humans, leading to many public health concerns and costs. Escherichia coli, a Gram-negative bacterial species, Enterococcus fecalis, a Gram-positive bacteria, and Candida albicans, a fungus, are only some microbial species that can infect humans. Antibacterial and antifungal medications have been developed to treat these infections successfully for years. However, extensive use of these antimicrobial medications has allowed some microbes to develop resistance to many of these treatments.

Drug resistant bacterial and fungal infections are becoming increasingly dangerous health problems. A recent study of United States academic hospitals indicates that nearly 1 in 20 patients are infected with methicillin-resistant Staphylococcus aureus (MRSA), the Gram-positive bacteria, and the rate of MRSA infections doubled between 2003 and 2008. Many of these infections are actually acquired within the hospital setting (nosocomial infections), leading to potentially life-threatening symptoms such as meningitis, septicemia, or devastating skin infections.

Thus, a need exists for new broad-spectrum antibacterial and antifungal drugs that can be used as a first-line defense, but that are also useful for microbes that are resistant to existing antimicrobial treatments.

### SUMMARY OF THE INVENTION

In one aspect, the invention relates to compounds of general formula (I):

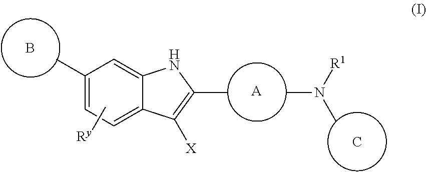

wherein

R¹ is selected from hydrogen and C₁₋₆ alkyl;

Ring A is selected from phenyl, thiophene and furan, wherein said phenyl, thiophene or furan may be optionally substituted with 1, 2, 3 or 4 R^{a} groups;

R^{a} is selected in each instance from hydrogen, halogen, C₁₋₆ alkyl, C₁₋₆ haloalkyl, C₁₋₆ alkoxy, C₁₋₆ haloalkoxy, amino, cyano, and nitro;

Ring B is selected from:

a) phenyl, thiophene and furan, substituted with at least one nitrogen-containing moiety, and further optionally substituted with one or more C₁₋₆ alkyl and/or C₁₋₆ alkoxy groups; and

b) a nitrogen-containing heterocyclyl, wherein said heterocyclyl may be optionally substituted with 1, 2, 3, 4 or 5 R^{b} groups;

R^{b} is selected in each instance from hydrogen, halogen, C₁₋₆ alkyl, C₋₆ haloalkyl, cyano, and R^{d};

R^{d} is chosen from carbocyclyl and heterocyclyl, wherein said carbocyclyl or heterocyclyl may be optionally substituted with 1, 2, 3, 4 or 5 substituents selected from halogen, C₁₋₆ alkyl, C₁₋₆ haloalkyl, C₁₋₆ alkoxy, C₁₋₆ haloalkoxy, nitro, amino, and cyano;

with the proviso that no more than one R^{b} may be R^{d};

Ring C is selected from heterocyclyl and carbocyclyl, wherein Ring C may be optionally substituted with 1, 2, 3, 4 or 5 R^{c} groups;

R^{c} is selected in each instance from hydrogen, halogen, C₁₋₆ alkyl, C₁₋₆ haloalkyl, C₁₋₆ alkoxy, C₁₋₆ haloalkoxy, cyano, nitro, amino, and R^{e};

R^{e} is chosen from carbocyclyl and heterocyclyl, wherein said carbocyclyl or heterocyclyl may be optionally substituted with 1, 2, 3, 4 or 5 substituents selected from halogen, C₁₋₆ alkyl, C₁₋₆ haloalkyl, C₁₋₆ alkoxy, C₁₋₆ haloalkoxy, cyano, nitro and amino;

with the proviso that no more than one R^{c} may be R^{e};

R^{y} represents one, two, or three groups individually selected from hydrogen, halogen, C₁₋₆ alkyl, C₁₋₆ haloalkyl, cyano, and nitro; and

X is selected from hydrogen, halogen, C₁₋₆ alkyl and C₁₋₆ haloalkyl.

The present invention provides, in a second aspect, a method of treating a bacterial infection in a subject in need thereof, comprising administering to the subject a therapeutically effective amount of a compound of formula (I) as disclosed herein above or a compound of formula (II)

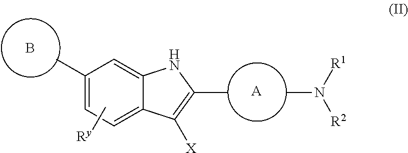

wherein

R¹ is selected from hydrogen and C₁₋₆ alkyl;

R² is hydrogen, C₁₋₆ alkyl, or a ring selected from heterocyclyl and carbocyclyl, wherein said ring may be optionally substituted with 1, 2, 3, 4 or 5 R groups;

R is selected in each instance from hydrogen, halogen, C₁₋₆ alkyl, C₁₋₆ haloalkyl, cyano, nitro, amino, carbocyclyl and heterocyclyl, wherein only one instance of R is carbocyclyl or heterocyclyl;

Ring A is selected from phenyl, thiophene and furan, wherein said phenyl, thiophene or furan may be optionally substituted with 1, 2, 3 or 4 R^{a} groups;

R^{a} is selected in each instance from hydrogen, halogen, C₁₋₆ alkyl, C₁₋₆ haloalkyl, C₁₋₆ alkoxy, C₁₋₆ haloalkoxy, amino, cyano, and nitro;

Ring B is selected from

a) phenyl, thiophene and furan, substituted with at least one nitrogen-containing moiety, and further optionally substituted with one or more C₁₋₆ alkyl and/or C₁₋₆ alkoxy groups; and

b) a nitrogen-containing heterocyclyl, wherein said heterocyclyl may be optionally substituted with 1, 2, 3, 4 or 5 R^{b} groups;

R^{b} is selected in each instance from hydrogen, halogen, C₁₋₆ alkyl, C₁₋₆ haloalkyl, cyano, and R^{d};

R^{d} is chosen from carbocyclyl and heterocyclyl, wherein said carbocyclyl or heterocyclyl may be optionally substituted with 1, 2, 3, 4 or 5 substituents selected from halogen, C₁₋₆ alkyl, C₁₋₆ haloalkyl, C₁₋₆ alkoxy, C₁₋₆ haloalkoxy, nitro, amino, and cyano;

with the proviso that no more than one R^{b} may be R^{d};

R^{y} represents one, two or three groups individually selected from hydrogen, halogen, C₁₋₆ alkyl, C₁₋₆ haloalkyl, C₁₋₆ alkoxy, C₁₋₆ haloalkoxy, amino, cyano and nitro; and

X is selected from hydrogen, halogen, C₁₋₆ alkyl and C₁₋₆ haloalkyl.

The present invention provides, in a third aspect, a method of treating a fungal infection in a subject in need thereof, comprising administering to the subject a therapeutically effective amount of a compound of formula (I) as disclosed herein above or a compound of formula (II).

The present invention provides, in a fourth aspect, a method of killing or inhibiting the growth of bacteria, comprising contacting the bacteria with a compound disclosed herein or a compound of formula (II)

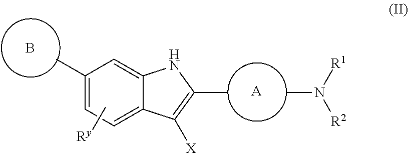

wherein

R¹ is selected from hydrogen and C₁₋₆ alkyl;

R² is hydrogen, C₁₋₆ alkyl, or a ring selected from heterocyclyl and carbocyclyl, wherein said ring may be optionally substituted with 1, 2, 3, 4 or 5 R groups;

R is selected in each instance from hydrogen, halogen, C₁₋₆ alkyl, C₁₋₆ haloalkyl, cyano, nitro, amino, carbocyclyl and heterocyclyl, wherein only one instance of R is carbocyclyl or heterocyclyl;

Ring A is selected from phenyl, thiophene and furan, wherein said phenyl, thiophene or furan may be optionally substituted with 1, 2, 3 or 4 R^{a} groups;

R^{a} is selected in each instance from hydrogen, halogen, C₁₋₆ alkyl, C₁₋₆ haloalkyl, C₁₋₆ alkoxy, C₁₋₆ haloalkoxy, amino, cyano, and nitro;

Ring B is selected from

a) phenyl, thiophene and furan, substituted with at least one nitrogen-containing moiety, and further optionally substituted with one or more C₁₋₆ alkyl and/or C₁₋₆ alkoxy groups; and

b) a nitrogen-containing heterocyclyl, wherein said heterocyclyl may be optionally substituted with 1, 2, 3, 4 or 5 R^{b} groups;

R^{b} is selected in each instance from hydrogen, halogen, C₁₋₆ alkyl, C₁₋₆ haloalkyl, cyano, and R^{d};

R^{d} is chosen from carbocyclyl and heterocyclyl, wherein said carbocyclyl or heterocyclyl may be optionally substituted with 1, 2, 3, 4 or 5 substituents selected from halogen, C₁₋₆ alkyl, C₁₋₆ haloalkyl, C₁₋₆ alkoxy, C₁₋₆ haloalkoxy, nitro, amino, and cyano;

with the proviso that no more than one R^{b} may be R^{d};

R^{y} represents one, two or three groups individually selected from hydrogen, halogen, C₁₋₆ alkyl, C₁₋₆ haloalkyl, C₁₋₆ alkoxy, C₁₋₆ haloalkoxy, amino, cyano and nitro; and

X is selected from hydrogen, halogen, C₁₋₆ alkyl and C₁₋₆ haloalkyl.

The present invention provides, in a fifth aspect, a method of killing or inhibiting the growth of fungus, comprising contacting the fungus with a compound disclosed herein or a compound of formula (II).

These and other objects, features and advantages of this invention will become apparent from the following detailed description of the various aspects of the invention taken in conjunction with the accompanying drawings.

### BRIEF DESCRIPTION OF THE DRAWINGS

FIG. 1 shows time kill curves for SM-1 bactericidal activity against Mycobacteria.

### DETAILED DESCRIPTION OF THE INVENTION

In one aspect, the invention relates to compounds having general formula (I)

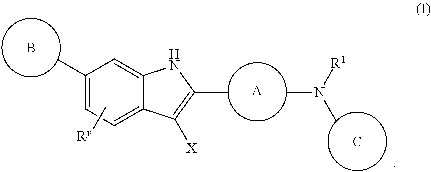

In some embodiments, R¹ is hydrogen. In some embodiments, R¹ is selected from a (C₁-C₆)alkyl. In some embodiments, R¹ is methyl.

In some embodiments, Ring A is phenyl. In some embodiments, Ring A is thiophene. In some embodiments, Ring A is furan.

In some embodiments, Ring A is optionally substituted with 1, 2, 3, or 4 R^{a} groups. R^{a} may be selected in each instance from hydrogen, halogen, C₁₋₆ alkyl, C₁₋₆ haloalkyl, C₁₋₆ alkoxy, C₁₋₆ haloalkoxy, amino, cyano, and nitro. To be perfectly clear, in some instances, Ring A may have only hydrogen substituents as R^{a} (i.e., be unsubstituted). For instance, in some embodiments, Ring A may be unsubstituted phenyl. In other non-limiting examples, R^{a} may be fluorine at 1, 2, 3, or 4 positions on Ring A, or R^{a} may be methyl at one position and cyano at another position.

In some embodiments, Ring B is phenyl substituted with at least one nitrogen-containing moiety. In some embodiments, Ring B is selected from thiophene substituted with at least one nitrogen-containing moiety. In some embodiments, Ring B is furan substituted with at least one nitrogen-containing moiety. In some embodiments, the nitrogen-containing moiety is amino. In other embodiments, the nitrogen-containing moiety is a nitrogen-containing monocycle. In some instances, the nitrogen-containing monocycle is morpholine or pyridine. In some embodiments when Ring B is phenyl, thiophene or furan, Ring B may be further optionally substituted with one or more C₁₋₆ alkyl and/or C₁₋₆ alkoxy groups. In some embodiments when Ring B is phenyl, thiophene or furan, Ring B may be further optionally substituted with one or more methyl and/or methoxy groups. In some embodiments, Ring B is a nitrogen-containing heterocyclyl. In some embodiments, Ring B is a nitrogen-containing heterocyclyl optionally substituted with 1, 2, 3, 4 or 5 R^{b} groups.

R^{b} may be selected in each instance from hydrogen, halogen, C₁₋₆ alkyl, C₁₋₆ haloalkyl, C₁₋₆ alkoxy, C₁₋₆ haloalkoxy, amino, cyano, and R^{d}. No more than one R^{b} may be R^{d}. To be perfectly clear, in some embodiments, Ring B may have only hydrogen substituents as R^{b} (i.e., be unsubstituted). For instance, in some embodiments, Ring B may be unsubstituted imidazoline. In another non-limiting example, Ring B may be substituted with one R^{d} group and one trifluoromethyl group.

In some embodiments, R^{d} is carbocyclyl. In other embodiments, R^{d} is heterocyclyl. In some embodiments, R^{d} is carbocyclyl optionally substituted with 1, 2, 3, 4 or 5 substituents selected from halogen, C₁₋₆ alkyl, C₁₋₆ haloalkyl, C₁₋₆ alkoxy, C₁₋₆ haloalkoxy, nitro, amino, and cyano. In other embodiments, R^{d} is heterocyclyl optionally substituted with 1, 2, 3, 4 or 5 substituents selected from halogen, C₁₋₆ alkyl, C₁₋₆ haloalkyl, nitro, amino, and cyano. To be perfectly clear, in some embodiments, R^{d} may be a carbocyclyl or heterocyclyl with no optional substitution (i.e., be unsubstituted). For instance, in some embodiments, R^{d} may be unsubstituted pyridine. In another non-limiting example, R^{d} may be phenyl substituted with one amino. In another non-limiting example, R^{d} may be phenyl substituted with fluorine at 1, 2, 3, or 4 positions.

In some embodiments, Ring C is heterocyclyl. In other embodiments, Ring C is a nitrogen-containing monocycle. In yet other embodiments, Ring C may be an aromatic nitrogen-containing monocycle such as imidazoline, pyridine, or pyrazine. In still other embodiments, Ring C may be a non-aromatic nitrogen-containing monocycle such as morpholine or piperidine. In some embodiments, Ring C is carbocyclyl. In other embodiments, Ring C is phenyl. In some embodiments, Ring C may be optionally substituted with 1, 2, 3, 4, or 5 R^{c} groups.

R^{c} may be selected in each instance from hydrogen, halogen, C₁₋₆ alkyl, C₁₋₆ haloalkyl, C₁₋₆ alkoxy, C₁₋₆ haloalkoxy, cyano, nitro, amino, and R^{e}. No more than one R^{c} may be R^{e}. To be perfectly clear, in some embodiments, Ring C may have only hydrogen substituents as R^{c} (i.e., be unsubstituted). For instance, in some embodiments, Ring C may be unsubstituted imidazoline. In another non-limiting example, Ring C may be substituted with one R^{c} group and one trifluoromethyl group.

In some embodiments, R^{e} is carbocyclyl. In other embodiments, R^{e} is heterocyclyl. In some embodiments, R^{e} is carbocyclyl optionally substituted with 1, 2, 3, 4 or 5 substituents selected from halogen, C₁₋₆ alkyl, C₁₋₆ haloalkyl, C₁₋₆ alkoxy, C₁₋₆ haloalkoxy, nitro, amino, and cyano. In other embodiments, R^{e} is heterocyclyl optionally substituted with 1, 2, 3, 4 or 5 substituents selected from halogen, C₁₋₆ alkyl, C₁₋₆ haloalkyl, nitro, amino, and cyano. To be perfectly clear, in some embodiments, R^{e} may be a carbocyclyl or heterocyclyl with no optional substitution (i.e., be unsubstituted). For instance, in some embodiments, R^{e} may be unsubstituted pyridine. In another non-limiting example, R^{e} may be phenyl substituted with one amino. In another non-limiting example, R^{e} may be phenyl substituted with fluorine at 1, 2, 3, or 4 positions.

In some embodiments, Ring C may be phenyl substituted with a nitrogen-containing monocycle. In some embodiments, the nitrogen-containing monocycle is unsubstituted. In other embodiments, Ring C may be phenyl substituted with amino or nitro. In other embodiments, Ring C may be unsubstituted phenyl. In still other embodiments, Ring C may be a nitrogen-containing monocycle and R^{c} is hydrogen or C₁₋₆ alkyl.

In some embodiments, R^{y} represents one, two or three groups individually selected in each instance from hydrogen, halogen, C₁₋₆ alkyl, C₁₋₆ haloalkyl, C₁₋₆ alkoxy, C₁₋₆ haloalkoxy, amino, cyano and nitro. In some embodiments, R^{y} represents hydrogen, halogen, methyl or trifluoromethyl. In other embodiments, R^{y} represents hydrogen. To be perfectly clear, in some embodiments, R^{y} may represent fluorine atoms at each of three positions, while in other embodiments, R^{y} may represent a methyl at one position and a cyano at another position.

In some embodiments, X is selected from hydrogen, halogen, C₁₋₆ alkyl and C₁₋₆ haloalkyl. In some embodiments, X is hydrogen. In other embodiments, X is halogen. In other embodiments, X is methyl. In still other embodiments, X is trifluoromethyl.

In some embodiments, R¹ is hydrogen or methyl; Ring A is optionally substituted phenyl; Ring B is optionally substituted imidazoline or phenyl substituted with amino and/or a nitrogen-containing monocycle; R^{y} is hydrogen, halogen, methyl or trifluoromethyl; X is hydrogen, halogen, methyl or trifluoromethyl; and Ring C is either: 1) phenyl and R^{c} is selected from hydrogen, a nitrogen-containing monocycle, amino and nitro; or 2) a nitrogen-containing monocycle and R^{c} is hydrogen or C₁₋₆ alkyl. In some of these embodiments, Ring B is phenyl substituted with amino, morpholino, and/or pyridinyl. In some of these embodiments, R^{c} is para-substituted.

In some embodiments, R¹ is hydrogen; Ring A is unsubstituted phenyl; Ring B is unsubstituted imidazoline; R^{y} is hydrogen; X is hydrogen; and Ring C is selected from 1) phenyl, wherein R^{c} is selected from hydrogen, a nitrogen-containing monocycle, amino and nitro; and 2) a nitrogen-containing monocycle, wherein R^{c} is hydrogen or C₁₋₆ alkyl.

In some embodiments, the compound is of formula

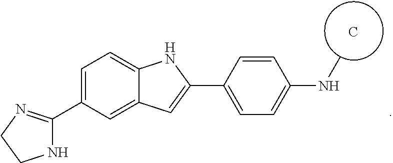

In some embodiments, the compound is of the formula above and R^{c} is substituted in the para position of Ring C.

In one aspect, the invention relates to a method of treating a bacterial infection in a subject in need thereof, comprising administering to the subject a therapeutically effective amount of at least one compound described herein or a pharmaceutical composition comprising at least one compound described herein. In one aspect, the invention relates to a method of treating a fungal infection in a subject in need thereof, comprising administering to the subject a therapeutically effective amount of at least one compound described herein or a pharmaceutical composition comprising a compound described herein. In another aspect, the invention relates to a method of treating a bacterial infection in a subject in need thereof, comprising administering to the subject a therapeutically effective amount of at least one compound of formula (II) or a pharmaceutical composition comprising a compound of formula (II):

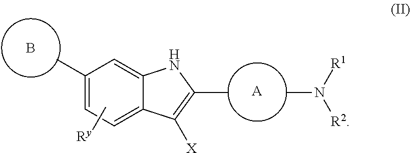

In another aspect, the invention relates to a method of treating a fungal infection in a subject in need thereof, comprising administering to the subject a therapeutically effective amount of at least one compound of formula (II) or a pharmaceutical composition comprising at least one compound of formula (II).

In formula (II), R¹, Ring A, R^{a}, Ring B, R^{b}, R^{d}, R^{y} and X are defined as above.

In some embodiments, R² is hydrogen. In other embodiments, R² is C₁₋₆ alkyl. In still other embodiments, R² is a heteroaryl. In other embodiments, R² is a heterocyclyl. In yet other embodiments, R² is a carbocyclyl. In some embodiments when R² is a heterocyclic or carbocyclic ring, the ring may be optionally substituted with 1, 2, 3, 4 or 5 R groups.

In some embodiments, R is selected in each instance from hydrogen, halogen, C₁₋₆ alkyl, C₁₋₆ haloalkyl, cyano, nitro, amino, carbocyclyl and heterocyclyl. Only one instance of R may be carbocyclyl or heterocyclyl. To be perfectly clear, as non-limiting examples, R may be hydrogen in all instances, or R may be phenyl at one position and fluorine at another position of the R² ring.

In some embodiments, the subject in need has a bacterial infection. In other embodiments, the bacterial infection is caused by gram negative bacteria. In some embodiments, the gram negative bacteria are Escherichia. In other embodiments, the gram negative bacteria are Klebsiella. In other embodiments, the gram negative bacteria are Pseudomonas. In some embodiments, the bacterial infection is caused by gram positive bacteria. In some embodiments, the gram positive bacteria are Staphylococcus. In some embodiments, the gram positive bacteria are Streptococcus. In some embodiments, the gram positive bacteria are Mycobacterium. In some embodiments, the gram positive bacteria are Enterococcus. It is important to note that the bacteria may be sensitive or resistant to already-existing drugs, such as vancomycin and methicillin.

In some embodiments, the subject in need has a fungal infection. In some embodiments, the fungal infection may be caused by a Candida species. In some embodiments, the fungal infection is caused by Candida glabrata. In some embodiments, the fungal infection is caused by Candida krusei. In some embodiments, the fungal infection is caused by Candida parapsilosis. In some embodiments, the fungal infection is caused by Candida albicans. It is important to note that the fungus may be sensitive or resistant to already-existing drugs and may be a multidrug resistant strain.

In some embodiments, the invention relates to a method of treating a bacterial infection in a subject in need thereof, comprising administering to the subject a therapeutically effective amount of the compound:

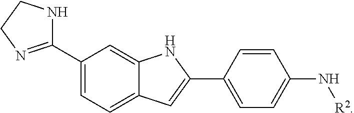

In some embodiments, the invention relates to a method of treating a fungal infection in a subject in need thereof, comprising administering to the subject a therapeutically effective amount of the compound:

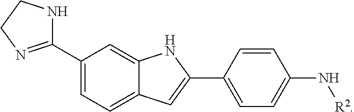

In other embodiments, the invention relates to a method of treating a bacterial infection in a subject in need thereof, comprising administering to the subject a therapeutically effective amount of at least one of the following compounds:

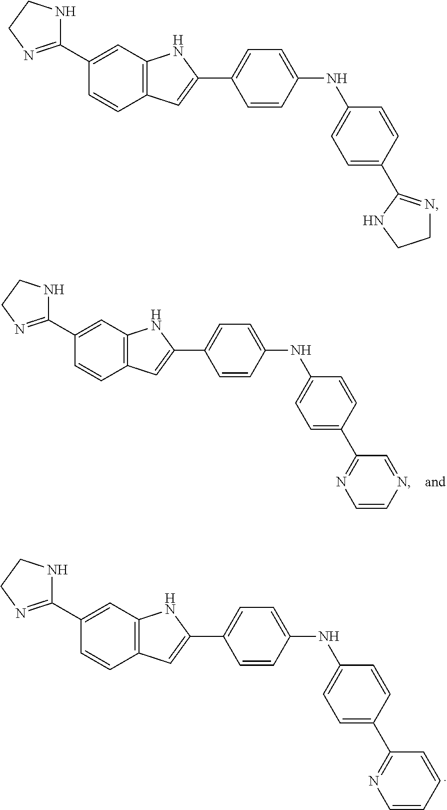 [SMILES: c1cc(C2=NCCN2)ccc1Nc1ccc(-c2cc3ccc(C4=NCCN4)cc3[nH]2)cc1 ; c1cnc(-c2ccc(Nc3ccc(-c4cc5ccc(C6=NCCN6)cc5[nH]4)cc3)cc2)cn1 ; "and" ; c1ccc(-c2ccc(Nc3ccc(-c4cc5ccc(C6=NCCN6)cc5[nH]4)cc3)cc2)nc1]

In some embodiments, the invention relates to a method of treating a fungal infection in a subject in need thereof, comprising administering to the subject a therapeutically effective amount of at least one of the following compounds:

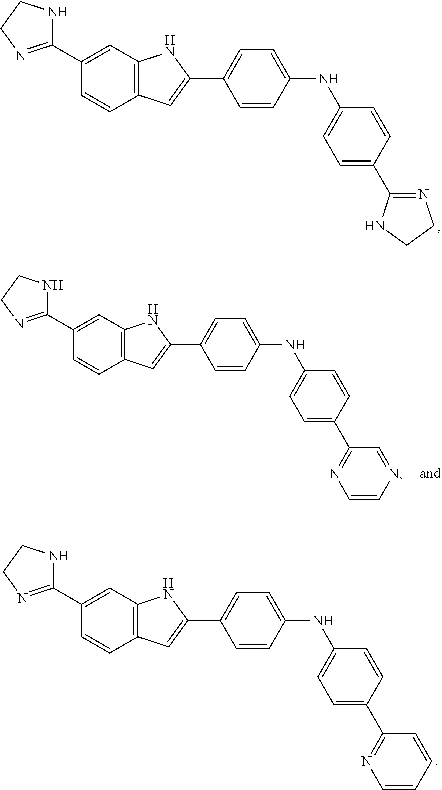 [SMILES: c1cc(C2=NCCN2)ccc1Nc1ccc(-c2cc3ccc(C4=NCCN4)cc3[nH]2)cc1 ; c1cnc(-c2ccc(Nc3ccc(-c4cc5ccc(C6=NCCN6)cc5[nH]4)cc3)cc2)cn1 ; c1ccc(-c2ccc(Nc3ccc(-c4cc5ccc(C6=NCCN6)cc5[nH]4)cc3)cc2)nc1]

In other embodiments, the invention relates to a method of treating a bacterial infection in a subject in need thereof, comprising administering to the subject a therapeutically effective amount of the compound:

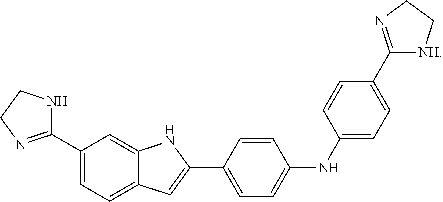 [SMILES: c1cc(C2=NCCN2)ccc1Nc1ccc(-c2cc3ccc(C4=NCCN4)cc3[nH]2)cc1]

In other embodiments, the invention relates to a method of treating a fungal infection in a subject in need thereof, comprising administering to the subject a therapeutically effective amount of the compound:

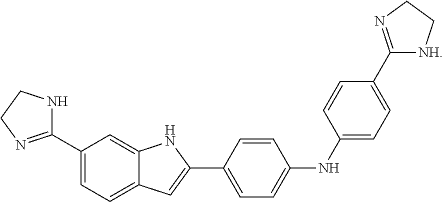 [SMILES: c1cc(C2=NCCN2)ccc1Nc1ccc(-c2cc3ccc(C4=NCCN4)cc3[nH]2)cc1]

In one aspect, the invention relates to a method of killing or inhibiting the growth of bacteria, comprising contacting the bacteria with a compound according to formula (I) or formula (II).

In one aspect, the invention relates to a method of killing or inhibiting the growth of fungus, comprising contacting the fungus with a compound according to formula (I) or formula (II).

In some embodiments, the invention relates to a method of killing or inhibiting the growth of bacteria, comprising contacting the bacteria with a compound:

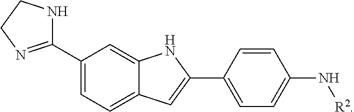

In some embodiments, the invention relates to a method of killing or inhibiting the growth of bacteria, comprising contacting the bacteria with a compound selected from

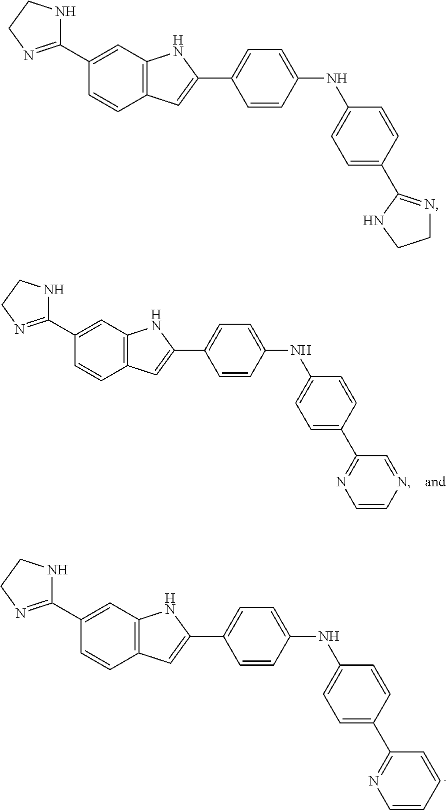 [SMILES: c1cc(C2=NCCN2)ccc1Nc1ccc(-c2cc3ccc(C4=NCCN4)cc3[nH]2)cc1 ; c1cnc(-c2ccc(Nc3ccc(-c4cc5ccc(C6=NCCN6)cc5[nH]4)cc3)cc2)cn1 ; "and" ; c1ccc(-c2ccc(Nc3ccc(-c4cc5ccc(C6=NCCN6)cc5[nH]4)cc3)cc2)nc1]

In other embodiments, the invention relates to a method of killing or inhibiting the growth of bacteria, comprising contacting the bacteria with a compound:

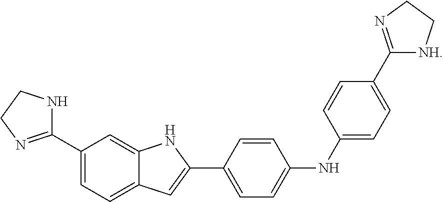 [SMILES: c1cc(C2=NCCN2)ccc1Nc1ccc(-c2cc3ccc(C4=NCCN4)cc3[nH]2)cc1]

In some embodiments, the invention relates to a method of killing or inhibiting the growth of fungus, comprising contacting the fungus with a compound:

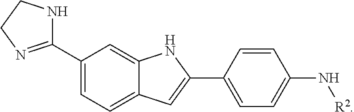

In some embodiments, the invention relates to a method of killing or inhibiting the growth of fungus, comprising contacting the fungus with a compound selected from

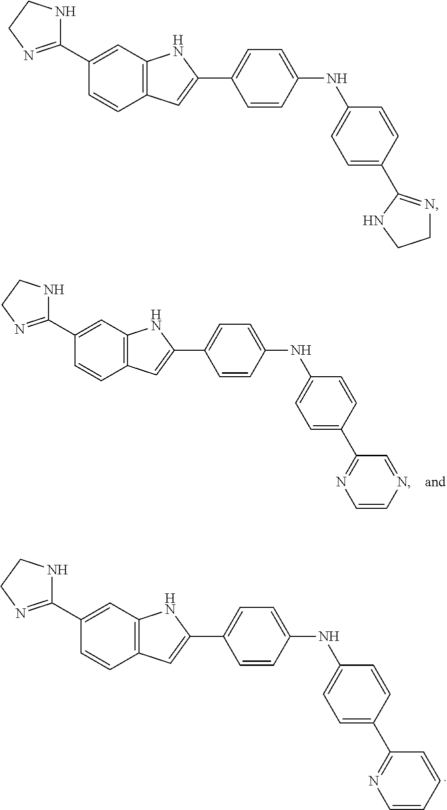 [SMILES: c1cc(C2=NCCN2)ccc1Nc1ccc(-c2cc3ccc(C4=NCCN4)cc3[nH]2)cc1 ; c1cnc(-c2ccc(Nc3ccc(-c4cc5ccc(C6=NCCN6)cc5[nH]4)cc3)cc2)cn1 ; "and" ; c1ccc(-c2ccc(Nc3ccc(-c4cc5ccc(C6=NCCN6)cc5[nH]4)cc3)cc2)nc1]

In other embodiments, the invention relates to a method of killing or inhibiting the growth of fungus, comprising contacting the fungus with a compound:

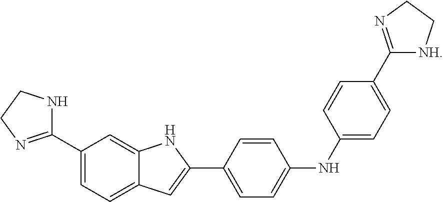 [SMILES: c1cc(C2=NCCN2)ccc1Nc1ccc(-c2cc3ccc(C4=NCCN4)cc3[nH]2)cc1]

In some embodiments, the bacteria are gram negative bacteria. In some embodiments, the gram negative bacteria are Escherichia. In some embodiments, the bacteria are Escherichia coli. In other embodiments, the gram negative bacteria are Klebsiella. In some embodiments, the bacteria are Klebsiella pneumoniae, including multidrug-resistant strains. In other embodiments, the gram negative bacteria are Pseudomonas. In some embodiments, the bacteria are Pseudomonas aeruginosa, including multidrug-resistant strains. In some embodiments, the bacteria are gram positive bacteria. In some embodiments, the gram positive bacteria are Staphylococcus. In some embodiments, the bacteria are Staphylococcus aureus, including methicillin-sensitive, methicillin-resistant and vancomycin-resistant strains. In some embodiments, the gram positive bacteria are Streptococcus. In some embodiments, the bacteria are Streptococcus pneumoniae, including drug-sensitive and drug-resistant strains. In some embodiments, the gram positive bacteria are Mycobacterium. In some embodiments, the bacteria are Mycobacterium tuberculosis. In some embodiments, the bacterial infection to be treated by this compound is an atypical mycobacterial infection. In some embodiments, the gram positive bacteria are Enterococcus. In some embodiments, the bacteria are Enterococcus faecalis, including vancomycin-resistant strains. In some embodiments, the bacteria are Enterococcus faecium, including vancomycin-resistant strains.

In some embodiments, the fungus is Candida, including those strains sensitive or resistant to one or more already-existing drugs. In some embodiments, the fungus is Candida albicans. In some embodiments, the fungus is Candida glabrata. In some embodiments, the fungus is Candida krusei. In some embodiments, the fungus is Candida parapsilosis.

It is to be understood that the bacterial or fungal infection may occur in the subject at various sites on the body. The site of infection often strain-specific. For instance, as non-limiting examples, a bacterial or fungal infection may affect the skin, the lungs, the sinuses, the blood, the genitals, the mucous membranes, or the brain.

For convenience and clarity certain terms employed in the specification, examples and claims are described herein.

Unless otherwise specified, alkyl (or alkylene) is intended to include linear, branched, or cyclic hydrocarbon structures and combinations thereof. A combination would be, for example, cyclopropylmethyl. Lower alkyl refers to alkyl groups of from 1 to 6 carbon atoms. Examples of lower alkyl groups include methyl, ethyl, propyl, isopropyl, butyl, s-and t-butyl and the like. Preferred alkyl groups are those of C₁₀ or below. Cycloalkyl is a subset of alkyl and includes cyclic hydrocarbon groups of from 3 to 8 carbon atoms. Examples of cycloalkyl groups include c-propyl, c-butyl, c-pentyl, norbornyl and the like.

C₁ to C₂₀ hydrocarbon includes alkyl, cycloalkyl, polycycloalkyl, alkenyl, alkynyl, aryl and combinations thereof. Examples include benzyl, phenyl, phenethyl, cyclohexylmethyl, adamantyl, camphoryl and naphthylethyl. Hydrocarbon refers to any substituent comprised of hydrogen and carbon as the only elemental constituents.

Unless otherwise specified, the term “carbocycle” is intended to include ring systems in which the ring atoms are all carbon but of any oxidation state. Thus (C₃-C₁₀) carbocycle refers to both non-aromatic and aromatic systems, including such systems as cyclopropane, benzene and cyclohexene; (C₈-C₁₂) carbopolycycle refers to such systems as norbornane, decalin, indane and naphthalene. Carbocycle, if not otherwise limited, refers to aromatic and non-aromatic monocycles, bicycles and polycycles.

Aryl and heteroaryl mean a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S. The aromatic 6- to 14-membered carbocyclic rings include, e.g., benzene, naphthalene, indane, tetralin, and fluorene and the 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole. As used herein aryl and heteroaryl refer to residues in which one or more rings are aromatic, but not all need be.

Heterocycle means a cycloalkyl or aryl residue in which one to two of the carbons is replaced by a heteroatom such as oxygen, nitrogen or sulfur. Heteroaryls form a subset of heterocycles. Non-limiting examples of heterocycles include pyrrolidine, pyrazole, pyrrole, imidazole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, pyrazine, thiophene, furan, oxazole, oxazoline, isoxazole, dioxane, tetrahydrofuran and the like.

Unless otherwise specified, alkoxy lkoxy or alkoxyl refers to groups of from 1 to 8 carbon atoms of a straight, branched or cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons. For the purpose of this application, alkoxy and lower alkoxy include methylenedioxy and ethylenedioxy.

The term “halogen” means fluorine, chlorine, bromine or iodine. In one embodiment, halogen may be fluorine or chlorine. The terms “haloalkyl” and “haloalkoxy” mean alkyl or alkoxy, respectively, substituted with one or more halogen atoms.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, 196, but without the restriction of 127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons has been replaced by sulfur or nitrogen, respectively. Examples of azaalkyl include ethylamino ethyl and amino hexyl.

The term “a nitrogen-containing moiety” is intended to encompass any substituent that contains nitrogen. Non-limiting examples include heterocyclic moieties (such as pyrrole, pyrroline, pyrrolidine, oxazole, oxazoline, oxazolidine, thiazole, thiazoline, thiazolidine, imidazole, imidazoline, imidazolidine, pyrazole, pyrazoline, pyrazolidine, isoxazole, isoxazoline, isoxazolidine, isothiazole, isothiazoline, isothiazolidine, oxadiazole, triazole, thiadiazole, pyridine, piperidine, morpholine, thiomorpholine, pyridazine, pyrimidine, pyrazine, piperazine, triazine, indolizine, indole, isoindole, indoline, indazole, benzimidazole, benzthiazole, purine, quinolizine, quinoline, isoquinoline, cinnoline, phthalazine, quinazoline, quinoxaline, naphthyridine, and the like) and acyclic moieties (such as amide, carboxamide, amine (primary, secondary and tertiary), imine, imide, azide, azo, cyanate, isocyanate, nitrate, nitrile, nitro, aniline, nitroso, and the like). A subset of these substituents includes amino and nitrogen-containing monocycles, such as imidazole.

As used herein, the term “optionally substituted” may be used interchangeably with “unsubstituted or substituted”. The term “substituted” refers to the replacement of one or more hydrogen atoms in a specified group with a specified radical. Substituted alkyl, aryl, cycloalkyl, heterocyclyl etc. refer to alkyl, aryl, cycloalkyl, or heterocyclyl wherein one or more H atoms in each residue are replaced with halogen, haloalkyl, alkyl, acyl, alkoxyalkyl, hydroxyloweralkyl, hydroxy, loweralkoxy, haloalkoxy, oxaalkyl, carboxy, nitro, amino, alkylamino, and/or dialkylamino. In one embodiment, 1, 2 or 3 hydrogen atoms are replaced with a specified radical. In the case of alkyl and cycloalkyl, more than three hydrogen atoms can be replaced by fluorine; indeed, all available hydrogen atoms could be replaced by fluorine.

The compounds described herein may contain, in a substituent R^{x}, double bonds and may also contain other centers of geometric asymmetry; unless specified otherwise, it is intended that the compounds include both E and Z geometric isomers. Likewise, all tautomeric forms are also intended to be included. The compounds may also contain, in a substituent R^{x}, one or more asymmetric centers and may thus give rise to enantiomers, diastereomers, and other stereoisomeric forms that may be defined, in terms of absolute stereochemistry, as (R)— or (S)—. The present invention is meant to include all such possible isomers, as well as their racemic and optically pure forms. Optically active (R)— and (S)— isomers may be prepared using chiral synthons or chiral reagents, or resolved using conventional techniques.

Substituents Rⁿ are generally defined when introduced and retain that definition throughout the specification and in all independent claims.

As used herein, and as would be understood by the person of skill in the art, the recitation of “a compound”—unless expressly further limited—is intended to include salts of that compound. Thus, for example, the recitation “a compound of formula I” as depicted above, which depicts a substituent COOH, would include salts in which the substituent is COO⁻M⁺, wherein M is any counterion. Similarly, formula I as depicted above depicts a substituent NH₂, and therefore would also include salts in which the substituent is NH₃⁺X⁻, wherein X is any counterion. The compounds may commonly exist as zwitterions, which are effectively internal salts. In a particular embodiment, the term “compound of formula I” refers to the compound or a pharmaceutically acceptable salt thereof. As used herein, and as would be understood by the person of skill in the art, the recitation of “a compound”—unless expressly further limited—is intended to include salts of that compound. In a particular embodiment, the term “compound of formula I” or “compound of formula II” refers to the compound or a pharmaceutically acceptable salt thereof.

The term “pharmaceutically acceptable salt” refers to salts whose counter ion derives from pharmaceutically acceptable non-toxic acids and bases. Suitable pharmaceutically acceptable acids for salts of the compounds of the present invention include, for example, acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. Suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

It will be recognized that the compounds of this invention can exist in radiolabeled form, i.e., the compounds may contain one or more atoms containing an atomic mass or mass number different from the atomic mass or mass number usually found in nature. Alternatively, a plurality of molecules of a single structure may include at least one atom that occurs in an isotopic ratio that is different from the isotopic ratio found in nature. Radioisotopes of hydrogen, carbon, phosphorous, fluorine, chlorine and iodine include ²H, ³H, ¹¹C, ¹³C, ¹⁴C, ¹⁵N, ³⁵S, ¹⁸F, ³⁶Cl, ¹²⁵I, ¹²⁴I and ¹³¹I respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Compounds that contain isotopes ¹¹C, ¹³N, ¹⁵O, ¹²⁴I and ¹⁸F are well suited for positron emission tomography. Radio labeled compounds of formulae I and II of this invention and prodrugs thereof can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

Although this invention is susceptible to embodiment in many different forms, preferred embodiments of the invention are shown. It should be understood, however, that the present disclosure is to be considered as an exemplification of the principles of this invention and is not intended to limit the invention to the embodiments illustrated. It may be found upon examination that certain members of the claimed genus are not patentable to the inventors in this application. In this event, subsequent exclusions of species from the compass of applicants' claims are to be considered artifacts of patent prosecution and not reflective of the inventors' concept or description of their invention; the invention encompasses all of the members of the genera I and II that are not already in the possession of the public.

While it may be possible for the compounds of formula I or II to be administered as the raw chemical, it is preferable to present them as a pharmaceutical composition. According to a further aspect, the present invention provides a pharmaceutical composition comprising a compound of formula I or II or a pharmaceutically acceptable salt thereof, together with one or more pharmaceutically acceptable carriers. The carrier(s) must be “acceptable” in the sense of being compatible with the other ingredients of the formulation and not deleterious to the recipient thereof. The compositions may be formulated for oral, topical or parenteral administration. For example, they may be given intravenously, intraarterially, subcutaneously, and directly into the CNS—either intrathecally or intracerebroventricularly.

Formulations include those suitable for oral, parenteral (including subcutaneous, intradermal, intramuscular, intravenous and intraarticular), rectal and topical (including dermal, buccal, sublingual and intraocular) administration. The compounds are preferably administered orally or by injection (intravenous or subcutaneous). The precise amount of compound administered to a patient will be the responsibility of the attendant physician. However, the dose employed will depend on a number of factors, including the age and sex of the patient, the precise disorder being treated, and its severity. Also, the route of administration may vary depending on the condition and its severity. The formulations may conveniently be presented in unit dosage form and may be prepared by any of the methods well known in the art of pharmacy. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose or multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological systems associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological systems of a disease, even though a diagnosis of this disease may not have been made.

Terminology related to “protecting”, “deprotecting” and “protected” functionalities occurs throughout this application. Such terminology is well understood by persons of skill in the art and is used in the context of processes that involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes of the invention, the person of ordinary skill can readily envision those groups that would be suitable as “protecting groups”. Suitable groups for that purpose are discussed in standard textbooks in the field of chemistry, such as Protective Groups in Organic Synthesis by T. W. Greene [John Wiley & Sons, New York, 1991], which is incorporated herein by reference.

A comprehensive list of abbreviations utilized by organic chemists appears in the first issue of each volume of the Journal of Organic Chemistry. The list, which is typically presented in a table entitled “Standard List of Abbreviations”, is incorporated herein by reference.

### Abbreviations

The following abbreviations and terms have the indicated meanings throughout:
Ac=acetyl
aq=aqueous
BINAP=2,2′-bis(diphenylphosphino)-1,1′-binaphthyl
Boc=t-butyloxy carbonyl
Bu=butyl
c-=cyclo
DCM=dichloromethane=methylene chloride=CH2Cl2
DMSO=dimethyl sulfoxide
EtOAc=ethyl acetate
EtOH=ethanol
h=hours
HOAc=acetic acid
HPLC=High-performance liquid chromatography
LCMS=Liquid chromatography-mass spectrometry
Me=methyl
MeOH=methanol
MIC=minimum inhibitory concentration
min.=minute
Pet. ether=petroleum ether
Ph=phenyl
PhOH=phenol
rac=racemic
rt (or RT)=room temperature
sat'd=saturated
s-=secondary
SDA=Sabourad dextrose agar
s-Phos=2-Dicyclohexylphosphino-2′,6′-dimethoxybiphenyl
t- or tert-=tertiary
TLC=thin layer chromatography

### Assays

A HTS screen was performed in M. smegmatis (mc2155) using a Beta-Galactosidase (B-gal) reporter gene. Compounds that inhibited B-Gal signal were then counterscreened using a disk diffusion assay in which each compound was assessed for its ability to inhibit growth of M. smegmatis, MRSA, or other bacteria. The “zone size” data given below refers to the size of the zone of inhibition produced by the compound. It is a reproducible measure of activity, but does not yield a specific drug concentration for comparison.

| | | | Zone Size |
|---|---|---|---|
| | | Zone Size | M. smegmatis |
| Cpd ID | Chemical Structure | MRSA (mm) | (mm) |
| SKI-1 | 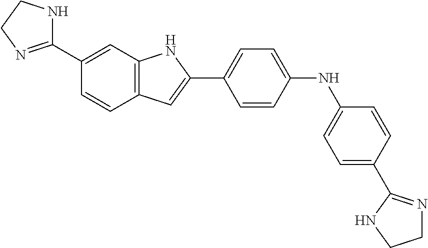 [SMILES: c1cc(C2=NCCN2)ccc1Nc1ccc(-c2cc3ccc(C4=NCCN4)cc3[nH]2)cc1] | 11 | 19.6 |
| SKI-2 | 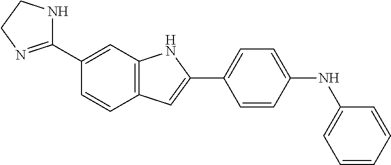 [SMILES: c1ccc(Nc2ccc(-c3cc4ccc(C5=NCCN5)cc4[nH]3)cc2)cc1] | 10 | 14.3 |
| SKI-4 | 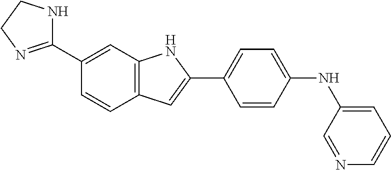 [SMILES: c1cncc(Nc2ccc(-c3cc4ccc(C5=NCCN5)cc4[nH]3)cc2)c1] | 14 | 27.6 |
| SKI-6 | 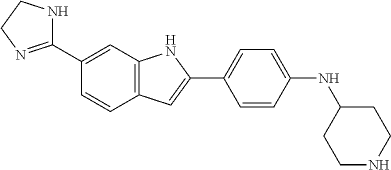 [SMILES: c1cc(-c2cc3ccc(C4=NCCN4)cc3[nH]2)ccc1NC1CCNCC1] | 15 | 16.3 |
| SKI-7 | 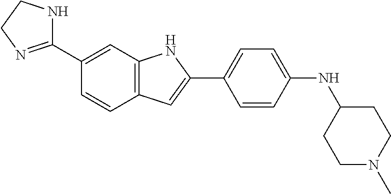 [SMILES: CN1CCC(Nc2ccc(-c3cc4ccc(C5=NCCN5)cc4[nH]3)cc2)CC1] | 19 | 30 |
| SKI-8 | 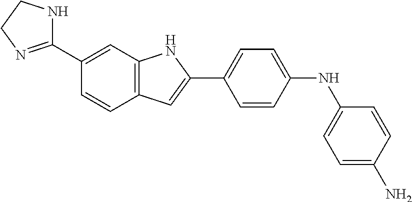 [SMILES: Nc1ccc(Nc2ccc(-c3cc4ccc(C5=NCCN5)cc4[nH]3)cc2)cc1] | 12 | 24 |
| SKI-10 | 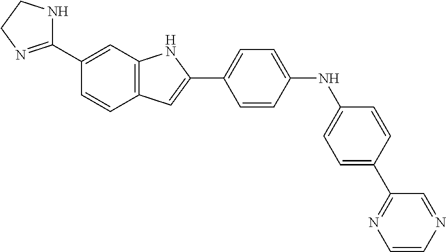 [SMILES: c1cnc(-c2ccc(Nc3ccc(-c4cc5ccc(C6=NCCN6)cc5[nH]4)cc3)cc2)cn1] | 11 | 14.3 |
| SKI-11 | 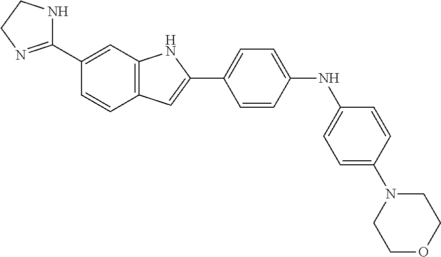 [SMILES: c1cc(-c2cc3ccc(C4=NCCN4)cc3[nH]2)ccc1Nc1ccc(N2CCOCC2)cc1] | 11 | 11 |
| SKI-12 | 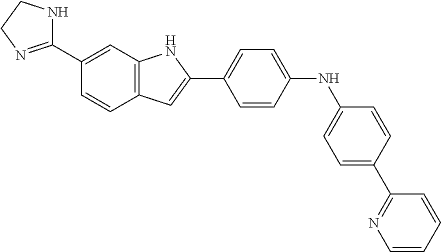 [SMILES: c1ccc(-c2ccc(Nc3ccc(-c4cc5ccc(C6=NCCN6)cc5[nH]4)cc3)cc2)nc1] | 10 | 12 |
| SKI-20 | 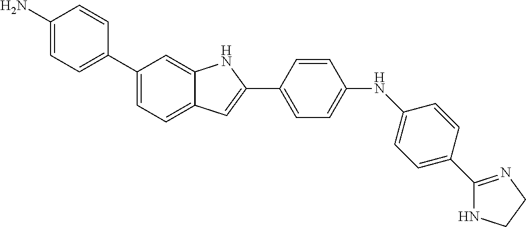 [SMILES: Nc1ccc(-c2ccc3cc(-c4ccc(Nc5ccc(C6=NCCN6)cc5)cc4)[nH]c3c2)cc1] | 9 | 8 |
| 398657 | 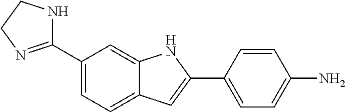 [SMILES: Nc1ccc(-c2cc3ccc(C4=NCCN4)cc3[nH]2)cc1] | 8.3 | 9.2 |

The “minimal inhibitory concentration” or “MIC” provides the actual concentration of drug that inhibits bacterial growth, either by causing growth arrest or cell death. In some cases, the inhibition of bacterial growth by either growth arrest or cell death was distinguished by testing the minimum bactericidal concentration (MBC), or the minimum concentration that will kill the tested organism. An antibiotic for which the MIC and MBC are similar are bactericidal whereas bacteriostatic antibiotics inhibit growth but do not kill bacteria, yielding a large difference between the MIC and MBC. Two sets of MIC data are presented from two separate test runs.

Antimicrobial Activity of SKI-1 against M. smegmatis and MRSA

| | | | | Average Zone | MIC | Average | MIC |
|---|---|---|---|---|---|---|---|
| IC50_381 | IC50_250 | IC50_251 | IC50_569 | Size MC2155 | Mc2155 | Zone Size | MRSA |
| (uM) | (uM) | (uM) | (uM) | (n = 3) | (μM) | MRSA | (μM) |
| 2.54 | 0.79 | 0.73 | 3.67 | 12.3 | 0.2 | 11 | 9.2 |

Recent testing against M. tuberculosis indicates that its activity is comparable to that observed for M. smegmatis.

In vitro Anti-bacterial & Anti-fungal Activity Assay: All test compounds were prepared as 6.4 mg/ml stock solutions in DMSO and further diluted according to the NCCLS M7-A6 (Page 5) document with sterile water or appropriate diluent. A working stock of 256 μg/ml was used to do 1:2 serial dilutions in 96-well plates. Final MIC concentrations range from 64 down to 0.12 μg/ml. The inoculums were prepared by making a direct sterile water suspension of isolated colonies from 18 to 24 hr agar plates for all organisms (Mueller Hinton, Blood Agar or SDA plates). Each bacterial suspension was adjusted to read between 0.09 and 0.11 absorbance at 620 nm. (0.5 McFarland Standard). These were further diluted 1/100 in appropriate broth for inoculating the 96-well plates. The C. albicans suspension was adjusted to read between 70 and 75% Transmittance at 530 nm and then diluted 1/500 in RPMI+MOPS broth. All aerobic bacterial and fungal plates were incubated for 18 hrs. at 35° C. S. pneumoniae plates were incubated in the presence of 5% CO₂. Plates were read using a Beckman Automated Plate Reader at 650 nm. Readings were confirmed by visual examination of plates. (See Table 1, Table 2, Table 3, and Table 4). In all of the assay and results descriptions, please note that “SM-1” and “356313” represent the same compound.

Many of the organism strains listed in Table 1, Table 2, Table 3, and Table 4 against which embodiments of the invention show activity are known to be drug-resistant. The listing of each strain with its known drug resistance is as follows: ATCC 33591 is MRSA; ATCC 700674 is Penicillin Resistant; ATCC 700221 is Vancomycin Resistant; and ATCC 29212 is Vancomycin resistant. BAA-39 is multi-drug resistant to the following drugs, as listed by ATCC: amoxicillin, cefaclor, cefuroxime, cephalexin, cephamandole, clindamycin, erythromycin, gentamicin, imipenem, oxacillin, penicillin, tetracycline, and tobramycin.

It is important to note that positive results against C. albicans are often indicative of broad spectrum antifungal activity, while negative results are not necessarily conclusory of a lack of activity against other fungus.

TABLE 1
Antimicrobial MIC (mcg/ml) Testing Results of SK Compounds against 14 Bacterial and Fungal Strains

| | S. aureus | S. aureus | S. aureus | S. pneumoniae | S. pneumoniae | E. faecalis | E. faecium |
|---|---|---|---|---|---|---|---|
| Compounds | ATCC | ATCC | BAA- | ATCC | ATCC | ATCC | ATCC |
| Tested | 29213 | 33591 | 39 | 6301 | 700674 | 29212 | 700221 |
| SKI-1 | 0.25 | 0.25 | 0.25 | ≦0.12 | ≦0.12 | 0.25 | ≦0.12 |
| SKI-2 | 8 | 4 | 4 | 2 | 4 | 2 | 2 |
| SKI-4 | 2 | 1 | 1 | ≦0.12 | 0.5 | 0.5 | 0.25 |
| SKI-6 | 8 | 8 | 8 | 32 | >64 | >64 | >64 |
| SKI-7 | 2 | 2 | 2 | 4 | 32 | 16 | 16 |
| SKI-8 | 16 | 8 | 16 | 8 | 32 | 8 | 4 |
| SKI-10 | 0.5 | 0.5 | 0.5 | ≦0.12 | ≦0.12 | 0.25 | ≦0.12 |
| SKI-11 | 8 | 4 | 4 | 4 | 16 | 4 | 1 |
| SKI-12 | 1 | 1 | 1 | ≦0.12 | ≦0.12 | | ≦0.12 |
| SKI-20 | >64 | >64 | >64 | 2 | 4 | 32 | 16 |
| | | | | | | | C. albicans |
| Compounds | E. faecium | E. coli | E. coli | K. pneumoniae | K. pneumoniae | P. aeruginosa | ATCC |
| Tested | CT-26 | ECM 1694 | ATCC 25922 | ATCC 13883 | ATCC 51503 | ATCC 9027 | 14053 |
| SKI-1 | ≦0.12 | 0.25 | 1 | 1 | 2 | 1 | 1 |
| SKI-2 | 2 | 2 | 16 | 32 | 64 | 64 | 16 |
| SKI-4 | 0.25 | ≦0.12 | 1 | >64 | >64 | >64 | >64 |
| SKI-6 | 64 | 4 | 32 | >64 | >64 | >64 | >64 |
| SKI-7 | 16 | 0.25 | 2 | >64 | >64 | >64 | 32 |
| SKI-8 | 4 | 2 | 32 | >64 | >64 | >64 | 64 |
| SKI-10 | ≦0.12 | 0.25 | 2 | 4 | 8 | 16 | 8 |
| SKI-11 | 1 | 1 | 16 | >64 | >64 | >64 | 32 |
| SKI-12 | ≦0.12 | 4 | 4 | 8 | 8 | 32 | 8 |
| SKI-20 | 8 | 64 | >64 | >64 | >64 | >64 | >64 |

TABLE 2
Antimicrobial MIC (mcg/ml) Testing Results of SK Compounds Against 14 Bacterial and Fungal Strains

| | S | S | S | S | S | E | E | | | | | | |
|---|---|---|---|---|---|---|---|---|---|---|---|---|---|
| | aureus | aureus | aureus | pneumonia | pneumonia | faecalis | faecium | | | | K | K | P |
| | ATCC | ATCC | BAA- | ATCC | ATCC | ATCC | ATCC | E | E. coli | E. coli | pneumonia | pneumonia | aeruginosa |
| | 29213 | 33591 | 39 | 6301 | 700674 | 29212 | 700221 | faecium | ECM | ATCC | ATCC | ATCC | ATCC |
| Cpd ID | MSSA | MRSA | MDR | PSSP | PRSP | VRE | VRE | CT-25 | 1693 | 25921 | 13883 | 51503 | 9026 |
| SKI-1 | 0.25 | 0.25 | 0.25 | 0.12 | 0.12 | 0.25 | 0.06 | 0.06 | 0.25 | 1 | 1 | 2 | 1 |
| SKI-2 | 8 | 4 | 4 | 2 | 4 | 2 | 2 | 2 | 2 | 16 | 32 | 64 | 64 |
| SKI-4 | 1 | 1 | 1 | 0.06 | 0.5 | 0.5 | 0.25 | 0.25 | 0.12 | 1 | 64 | 64 | 64 |
| SKI-6 | 8 | 8 | 8 | 32 | 64 | 64 | 64 | 64 | 4 | 32 | 64 | 64 | 64 |
| SKI-7 | 2 | 2 | 2 | 4 | 32 | 16 | 16 | 16 | 0.25 | 2 | 64 | 64 | 64 |
| SKI-8 | 16 | 8 | 16 | 8 | 32 | 8 | 4 | 4 | 2 | 32 | 64 | 64 | 64 |
| SKI-10 | 0.5 | 0.5 | 0.5 | 0.12 | 0.12 | 0.25 | 0.12 | 0.12 | 0.25 | 2 | 4 | 8 | 16 |
| SKI-11 | 8 | 4 | 4 | 4 | 16 | 4 | 1 | 1 | 1 | 16 | 64 | 64 | 64 |
| SKI-12 | 1 | 1 | 1 | 0.12 | 0.12 | 0.25 | 0.12 | 0.12 | 4 | 4 | 8 | 8 | 32 |
| SKI-20 | 64 | 64 | 64 | 2 | 4 | 32 | 16 | 8 | 64 | 64 | 64 | 64 | 64 |

(numbers are broth dilution MIC in ug/ml)

TABLE 3
Antimicrobial MIC (mcg/ml) Testing Results of Select SK Compounds against Gram-positive
Organisms and E. Coli ECM 1694 (starting at 4 μg/ml)

| | S. | S. | S. | S. | S. | E. | E. | | |
|---|---|---|---|---|---|---|---|---|---|
| | aureus | aureus | aureus | pneumoniae | pneumoniae | faecalis | faecium | E. | E. coli |
| Compounds | ATCC | ATCC | BAA- | ATCC | ATCC | ATCC | ATCC | faecium | ECM |
| Tested | 29213 | 33591 | 39 | 6301 | 700674 | 29212 | 700221 | CT-26 | 1694 |
| SKI-1 | 0.25 | 0.25 | 0.25 | 0.12 | 0.12 | 0.25 | 0.06 | 0.06 | 0.5 |
| SKI-4 | 1 | 1 | 1 | 0.06 | 0.5 | 0.5 | 0.25 | 0.25 | 0.12 |
| SKI-10 | 0.5 | 0.5 | 0.5 | 0.12 | 0.12 | 0.25 | 0.12 | 0.12 | 0.25 |
| SKI-12 | 1 | 1 | 1 | 0.12 | 0.12 | 0.25 | 0.12 | 0.12 | 4 |
| CPLX | 0.25 | 0.12 | >4 | 0.5 | 2 | 1 | >4 | >4 | ≦0.008 |
| IMP | 0.016 | >4 | 1 | ≦0.008 | 0.06 | 1 | >4 | >4 | 0.5 |

TABLE 4
Antimocrobial MIC (mcg/ml) Testing Results of Select SK Compounds Against Drug Sensitive and
Resistant Gram Positive Pathogens

| | MSSA | MRSA | MDR | PSSP | PRSP | VRE | VRE |
|---|---|---|---|---|---|---|---|
| Compounds | S. aureus | S. aureus | S. aureus | S. pneumoniae | S. pneumoniae | E. faecalis | E. faecium |
| Tested | ATCC 29213 | ATCC 33591 | BAA-39 | ATCC 6301 | ATCC 700674 | ATCC 29212 | ATCC 700221 |
| 356313 | 0.25 | 0.12 | 0.25 | 0.12 | 0.03 | 0.12 | 0.03 |
| CPLX | 0.12 | 0.015 | 2 | 0.12 | 0.06 | 0.25 | >4 |
| IMP | 0.06 | >4 | 4 | 0.015 | 0.5 | 4 | >4 |
| | Compounds | E. faecium | E. coli | E. coli | K. pneumoniae | K. pneumoniae | P. aeruginosa |
| | Tested | CT-26 | ATCC 25922 | ECM 1694 | ATCC 13883 | ATCC 51503 | ATCC 9027 |
| | 356313 | 0.015 | 0.5 | 0.25 | 0.5 | 1 | 0.5 |
| | CPLX | >4 | ≦0.008 | ≦0.008 | ≦0.008 | 0.06 | 0.03 |
| | IMP | >4 | 1 | 2 | 2 | 2 | 4 |

[Values in mg/ml by broth dilution MIC according to CLSI standards (NAEJA)]

Thigh Infection Model Assay in Neutropenic Mice: The efficacy of SKI-1 (also referenced as 356313 below) was evaluated at 10 and 20 mg/kg doses against methicillin resistant Staphylococcus aureus (MRSA) and Streptococcus pneumoniae infections in a neutropenic mouse thigh model. The mice were infected with MRSA or S. pneumoniae in the thighs and the treatments were given by two intrperitoneal administrations at six (6) hours apart after two hours of the infection. The bacterial tissue burdens in thighs were determined after 24 hours of treatment. The effects of SKI-1 at two dose levels, 10 and 20 mg/kg, were tested against MRSA and S. pneumoniae infections in thighs of immuno-compromised CD1 mice. Mice were rendered neutropenic by cyclophosphamide injections and were infected with the organism into the thigh muscles. The mice were treated two (2) and eight (8) hours post-infection by intraperitoneal administration of SKI-1 and vancomycin at 100 mg/kg doses by subcutaneous injection. The tissues were collected after 24 hours and processed for bacterial tissue burdens. The mice were grouped as below (Table 5):

TABLE 5

| | | Route of | | Day-4 | Day-1 | Day 0 | | | Day 1 | Day 2 |
|---|---|---|---|---|---|---|---|---|---|---|
| | | admin- | # of | Cyclophosphamide | Cyclophosphamide | Infection, | 2 hr post- | 8 hr post- | 26 hr post- | 24 hr post- |
| Group | Treatment | istration | mice | (Cpd) injection (IP) | (Cpd) injection (IP) | @ 9am | infection | infection | infection | tissue |
| 1 | Control | IP | 8 | Cpd, 150 mg/kg | Cpd, 100 mg/kg | Infection | Dosing | Dosing | Tissue | Plate |
| | (Vehicle) | | | | | | | | processing | counting |
| 2 | Vancomycin, | SC | 8 | Cpd, 150 mg/kg | Cpd, 100 mg/kg | Infection | Dosing | Dosing | Tissue | Plate |
| | 100 mg/kg, BID | | | | | | | | processing | counting |
| 3 | 356313, | IP | 8 | Cpd, 150 mg/kg | Cpd, 100 mg/kg | Infection | Dosing | Dosing | Tissue | Plate |
| | 10 mg/kg, BID | | | | | | | | processing | counting |
| 4 | 356313, | IP | 8 | Cpd, 150 mg/kg | Cpd, 100 mg/kg | Infection | Dosing | Dosing | Tissue | Plate |
| | 20 mg/kg, BID | | | | | | | | processing | counting |
| | | | 32 | | | | | | | |

The study was performed with methicillin-resistant S. aureus (ATCC 33591) and S. pneumoniae (ATCC 6303) (American Type Culture Collection, Rockville, Md.). The organisms were grown in Mueller-Hinton agar (MHA) and Brain Heart Infusion agar (BHIA) plates, respectively. For growth in liquid media, cation adjusted Mueller-Hinton broth (MHB) and Brain Heart Infusion (BHIB) broth were used, respectively. Sixty-four (64) male 6-8 week old CD1 mice (20-22 gm each, Charles River, Canada) were used in this study and 32 mice were used for each infection group. The mice were provided with sterile rodent chow diet and free flowing water. They were monitored daily during the experiment and clinical symptoms such as condition of the fur coat, the amount of facial grooming, and the degree of physical and respiratory activities of each animal were recorded on case report forms. S. aureus (MRSA) (ATCC33591) and S. pneumoniae (ATCC 6303) were grown fresh from the frozen stock (at −80° C.) onto Muller Hinton Agar (MHA) or Brain Heart Infusion agar (BHIA) plates at 37° C. After checking the purity, few pure single colonies were picked and inoculated in Muller Hinton broth (MHB) and Brain Heart Infusion broth (BHIB) and grown overnight to a late log phase (around 12 hours) in a shaking incubator at 37° C. The culture was centrifuged at 4000 rpm for 10 minutes at 4° C. and the cells were resuspended in sterile normal saline (0.9% Nacl). The cells were washed twice similarly by centrifuging and resuspending in saline. The final inoculums were prepared to 1 OD580 (optical density at 580 nm spectrophotometer reading), which were equal to a known number of bacteria (from previous expt.), and then diluted further to 5×10⁶ cfu/ml. A volume of 0.1 ml of the inoculums was injected into one thigh of each mouse. The mice were rendered neutropenic by injecting cyclophosphamide (Sigma, Canada) at 150 gm/kg and 100 mg/kg by intraperitoneal (IP) route on four (Day −4) and one day (Day −1) before the day of infection (Day 0). On Day 0, the mice were injected with 0.1 ml of the inoculums, as described above, into one of the thighs each mouse. Each mouse was restrained and maintained by one person, while another person cleaned the thigh with 70% alcohol and injected the inoculums into deep muscle of the thigh. The thigh was cleaned again with 70% alcohol and the mouse was returned into the cage. The mice were treated two hours after the infection, as detailed in Table 5. Vehicle and 356313 (SM-1) were administered by intraperitoneal injection, but vancomycin was injected by subcutaneous injection. The treatment was given twice at 6 hours apart and mice were observed for 24 hours. The mice were euthanized humanely after 24 hours post-infection by carbon dioxide inhalation and the infected thighs were excised aseptically. The muscles from the thighs were dissected and collected in a round-bottomed tube containing 3 ml sterile saline. The tissues were homogenized by Brinkmann Polytron PT300 homogenizer at 22-24K rpm and the resulting homogenates were serially ten-fold diluted (six times) in sterile saline. One hundred microlitres (100 μL) of each dilution was plated onto MHA or BHIA plates in duplicates and the plates were incubated at 37° C. for 24-48 hours. The colonies were counted and the colony forming units for each thigh (CFU/3 ml) were determined and log10 of the counts were calculated.

The efficacies of treatments were analyzed by comparing the data with the counts of vehicle group by using the built-in statistical tests of GraphPad Prism (version 5) and P-values of the groups were determined.

The effects of treatments have been evaluated at 10 and 20 mg/kg. Vancomycin was used as positive control and to compare the test compounds. The result is summarized in the graphs and tables below:

TABLE 6
Efficacy of 356313 (SKI-1) treatments against MRSA infection in mice

| | Mean | SD | Log |
|---|---|---|---|
| Group | (log10/thigh) | (log10/thigh) | reduction |
| Control (Vehicle) | 8.4488 | 0.1251 | |
| Vancomycin 100 mg/kg BID | 5.0033 | 0.7898 | 3.4455 |
| 356313, 10 mg/kg, BID | 8.3106 | 0.2933 | 0.1382 |
| 356313, 20 mg/kg, BID | 7.7428 | 0.4573 | 0.7060 |

TABLE 7
Efficacy of 356313 (SKI-1) treatments against
S. pneumoniae infection in mice

| | Mean | SD | Log |
|---|---|---|---|
| Group | (log10/thigh) | (log10/thigh) | reduction |
| Control (Vehicle) | 9.1127 | 0.1203 | |
| Vancomycin, 100 mg/kg, BID | 3.4862 | 1.4899 | 5.6265 |
| 356313, 10 mg/kg, BID | 6.7143 | 1.0109 | 2.3984 |
| 356313, 20 mg/kg, BID | 5.6260 | 1.3244 | 3.4867 |

For MRSA infection, the 356313 (SKI-1) treatments at 10 and 20 mg/kg (compared to the vehicle treatment) reduced bacterial tissue burdens 0.14 and 0.71 Logs, respectively, after 24 hours. For the same duration, Vancomycin at 100 mg/kg reduced the tissue burdens 3.45 logs. For S. pneumoniae, the reductions in tissue burden were 2.40 and 3.49 logs for treatments at 10 and 20 mg/kg, respectively, and for vancomycin, the reduction was 5.63 logs.

TABLE 8
Statistical analysis of 356313 (SKI-1)
treatments against MRSA infection in mice
Statistical analysis of 356313 against MRSA in a thigh model:

| Unpaired t test | Vancomycin, | 356313, | 356313, |
|---|---|---|---|
| (against Vehicle group) | 100 mg/kg | 10 mg/kg | 20 mg/kg |
| P value | <0.0001 | 0.2404 | 0.0009 |
| P value summary | **** | ns | *** |
| Are means signif. | Yes | No | Yes |
| different? (P < 0.05) | | | |
| One- or two-tailed p value? | Two-tailed | Two-tailed | Two-tailed |
| t, df | t = 12.19 df = 14 | t = 1.226 | t = 4.212 |
| | | df = 14 | df = 14 |

TABLE 9
Statistical analysis of 356313 (SKI-1) treatments
against S. pneumoniae infection in mice:
Statistical analysis of 356313 against S. pneumoniae in a thigh model:

| Unpaired t test | Vancomycin, | 356313, | 356313, |
|---|---|---|---|
| (against Vehicle group) | 100 mg/kg | 10 mg/kg | 20 mg/kg |
| P value | <0.0001 | <0.0001 | <0.0001 |
| P value summary | **** | **** | **** |
| Are means signif. | Yes | Yes | Yes |
| different? (P < 0.05) | | | |
| One- or two-tailed p value? | Two-tailed | Two-tailed | Two-tailed |
| t, df | t = 10.92 df = 11 | t = 6.663 | t = 7.416 |
| | | df = 14 | df = 14 |

Compared to the vehicle group, SKI-1 reduced the tissue burden significantly by 20 mg/kg treatments for both MRSA and S. pneumoniae (P=0.0009 and P=0.0001, respectively), but SKI-1 at 10 mg/kg reduced the tissue burden significantly (P<000.1) for S. pneumoniae only.

In-vivo Efficacy of SKI-1 Against MRSA and E. Coli Septicemia Model in Neutropenic CD1 Mice: SKI-1 was evaluated at 10 and 20 mg/kg doses against methicillin resistant Staphylococcus aureus (MRSA) and Escherichia coli infections in a mouse neutropenic septicemia survival model. The treatments with SKI-1 at 10 and 20 mg/kg were seen to be effective (P=0.0495 and P=0.008, respectively) against MRSA infection, but not against E. coli infection (P=0.5127 and P=0.0719 for 356313 (SKI-1) at 10 and 20 mg/kg, respectively).

The study was set up in a neutropenic mouse survival model and the effect of compound 356313 (SKI-1) was tested in the model at two dose levels, i.e., 10 and 20 mg/kg against MRSA and E. coli infections. The mice were rendered neutropenic by cyclophosphamide injections and were infected with the organisms by injection in to the lateral tail veins. The mice were treated at two (2) and eight (8) hours post-infection by intraperitoneal administration of 356313 (SKI-1) and vehicle. Vancomycin at 100 mg/kg and Gentamicin at 10 mg/kg were used as controls and injected subcutaneously for the same duration. The survival of the mice was observed for 7 days. The mice were grouped as below:

| | | Route of | | Day-4 | Day-1 | Day 0 | | | |
|---|---|---|---|---|---|---|---|---|---|
| | | admin- | # of | Cyclophosphamide | Cyclophosphamide | Infection, | 2 hr post- | 8 hr post- | Day 1-7 |
| Group | Treatment | istration | mice | (Cpd) injection (IP) | (Cpd) injection (IP) | @ 9am | infection | infection | Observation |

In-vivo Efficacy of 356313 against S. aureus septicemia in a neutropenic model in CD1 mice

| | | | | | | | | | |
|---|---|---|---|---|---|---|---|---|---|
| 1 | Control | IP | 5 | Cpd, 150 mg/kg | Cpd, 150 mg/kg | Infection | Dosing | Dosing | Observation |
| | (Vehicle) | | | | | | | | |
| 2 | Gentamicin, | SC | 5 | Cpd, 150 mg/kg | Cpd, 150 mg/kg | Infection | Dosing | Dosing | Observation |
| | 10 mg/kg, BID | | | | | | | | |
| 3 | 356313, | IP | 5 | Cpd, 150 mg/kg | Cpd, 150 mg/kg | Infection | Dosing | Dosing | Observation |
| | 10 mg/kg, BID | | | | | | | | |
| 4 | 356313, | IP | 5 | Cpd, 150 mg/kg | Cpd, 150 mg/kg | Infection | Dosing | Dosing | Observation |
| | 20 mg/kg, BID | | | | | | | | |

In-vivo Efficacy of 356313 against E. coli septicemia in a neutropenic model in CD1 mice

| | | | | | | | | | |
|---|---|---|---|---|---|---|---|---|---|
| 1 | Control | IP | 5 | Cpd, 150 mg/kg | Cpd, 150 mg/kg | Infection | Dosing | Dosing | Observation |
| | (Vehicle) | | | | | | | | |
| 2 | Gentamicin, | SC | 5 | Cpd, 150 mg/kg | Cpd, 150 mg/kg | Infection | Dosing | Dosing | Observation |
| | 10 mg/kg, BID | | | | | | | | |
| 3 | 356313, | IP | 5 | Cpd, 150 mg/kg | Cpd, 150 mg/kg | Infection | Dosing | Dosing | Observation |
| | 10 mg/kg, BID | | | | | | | | |
| 4 | 356313, | IP | 5 | Cpd, 150 mg/kg | Cpd, 150 mg/kg | Infection | Dosing | Dosing | Observation |
| | 20 mg/kg, BID | | | | | | | | |

Animals: Forty (40) male 6-8 week old CD1 mice (20-22 gm each) were used in this study and 20 mice were used for each infection group. The mice were purchased from Charles River (Canada) and housed in 5 mice per cage. The mice were provided with sterile rodent chow diet and free flowing water. They were monitored daily during the experiment and clinical symptoms such as condition of the fur coat, the amount of facial grooming, and the degree of physical and respiratory activities of each animal were recorded on the case report forms.

Inoculums preparation: S. aureus (MRSA) (ATCC33591) and E. coli (ATCC 25922) were grown fresh from frozen stock (at −80° C.) onto Muller Hinton Agar (MHA) plates at 37° C. After checking the purity, few pure single colonies were picked and inoculated in Muller Hinton broth (MHB) and grown overnight to a late log phase (around 12 hours) in a shaking incubator at 37° C. The culture was centrifuged at 4000 rpm for 10 minutes at 4° C. and the cells were resuspended in sterile normal saline (0.9% Nacl). The cells were washed twice similarly by centrifuging and resuspending in saline. The final inoculums were prepared to 1 optical density at 580 nm spectrophotometric reading, which were equal to a known number of bacterial counts, and then diluted further and a volume of 0.1 ml of the inoculum was injected into one thigh of each mouse so that each mouse would receive 1×10⁷ cfu.

Neutropenic Mouse Thigh Infection Model: The mice were rendered neutropenic by injecting cyclophosphamide (Sigma, Canada) at 150 gm/kg and 100 mg/kg by intraperitoneal (IP) route on four (Day −4) and one (Day −1) days before the day of infection (Day 0). On Day 0, the mice were injected with 0.1 ml of the inoculums, as described above, into the tail veins of the mice.

Treatment: The mice were treated two hours after the infection, as detailed in the tables above. 356313 (SM-1) and vehicle were administered by intraperitoneal injection, but vancomycin was injected by subcutaneous injection. The treatment was given twice at 6 hours apart and mice were observed for any unexpected reaction or change in the clinical symptoms.

Statistics: The survival trends were analyzed and the efficacies of 356313 (SKI-1) treatments were determined. The survival curves of the treatment groups were compared with the control group by using the built-in survival test of GraphPad Prism (version 5) and the P-values were determined.

The efficacies of 356313 (SKI-1) treatments, at 10 and 20 mg/kg, were determined by the length of survival, which was expressed as the percent survival for each group. The survival trends of the groups have been summarized in the tables and graphs below:

TABLE 10
Efficacy of 356313 (SKI-1) treatments against MRSA infection in mice
In-vivo Efficacy of 356313 against S. aureus in a neutropenic model
in CD1 mice

| Group/Percent | | | | | | | |
|---|---|---|---|---|---|---|---|
| Survival | Day 1 | Day 2 | Day 3 | Day 4 | Day 5 | Day 6 | Day 7 |
| Control (Vehicle) | 100 | 40 | 0 | 0 | 0 | 0 | 0 |
| Vancomycin, | 100 | 100 | 100 | 80 | 80 | 80 | 80 |
| 100 mg/kg, BID | | | | | | | |
| 356313, | 100 | 100 | 0 | 0 | 0 | 0 | 0 |
| 10 mg/kg, BID | | | | | | | |
| 356313, | 100 | 100 | 80 | 0 | 0 | 0 | 0 |
| 20 mg/kg, BID | | | | | | | |

TABLE 11
Efficacy of 356313 (SKI-1) treatments against E. coli infectionin mice
In-vivo Efficacy of 356313 against E. coli in a neutropenic model
in CD1 mice

| Group/Percent | | | | | | | |
|---|---|---|---|---|---|---|---|
| Survival | Day 1 | Day 2 | Day 3 | Day 4 | Day 5 | Day 6 | Day 7 |
| Control (Vehicle) | 100 | 0 | 0 | 0 | 0 | 0 | 0 |
| Gentamicin, | 100 | 100 | 80 | 60 | 60 | 60 | 60 |
| 10 mg/kg, BID | | | | | | | |
| 356313, | 100 | 60 | 0 | 0 | 0 | 0 | 0 |
| 10 mg/kg, BID | | | | | | | |
| 356313, | 100 | 80 | 60 | 0 | 0 | 0 | 0 |
| 20 mg/kg, BID | | | | | | | |

As shown above, all the mice of the vehicle groups for both the infections died by Day 2 or 3; but for Vancomycin or Gentamicin groups, 80% and 60% of the mice survived until the end of the experiment. For 356313 (SKI-1) treatment at 10 mg/kg, all the mice died by Day 3 in both MRSA and E. coli infections; whereas the treatment at 20 mg/kg prolonged the survival one day more, i.e., the mice died by Day 4, in both infection experiments. The survival trends of the mice in different groups were analyzed. Statistical analysis compared to the vehicle group revealed that the survival trends for 356313 (SKI-1) treatments at 10 and 20 mg/kg were slightly and moderately significant (P=0.0495 and P=0.008), respectively, for MRSA infection. On the contrary, same treatments did not show any significant difference (P=0.5127 and P=0.0719, respectively) in E. coli infection. The survival trends for Vancomycin and Gentamicin against the vehicle group were highly effective (P=0.0023 and P=0.0047, respectively).

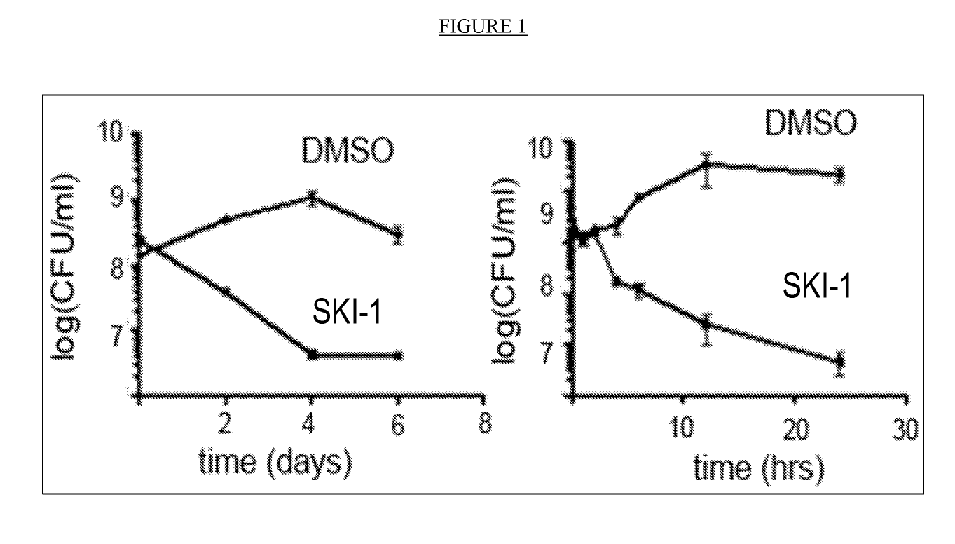

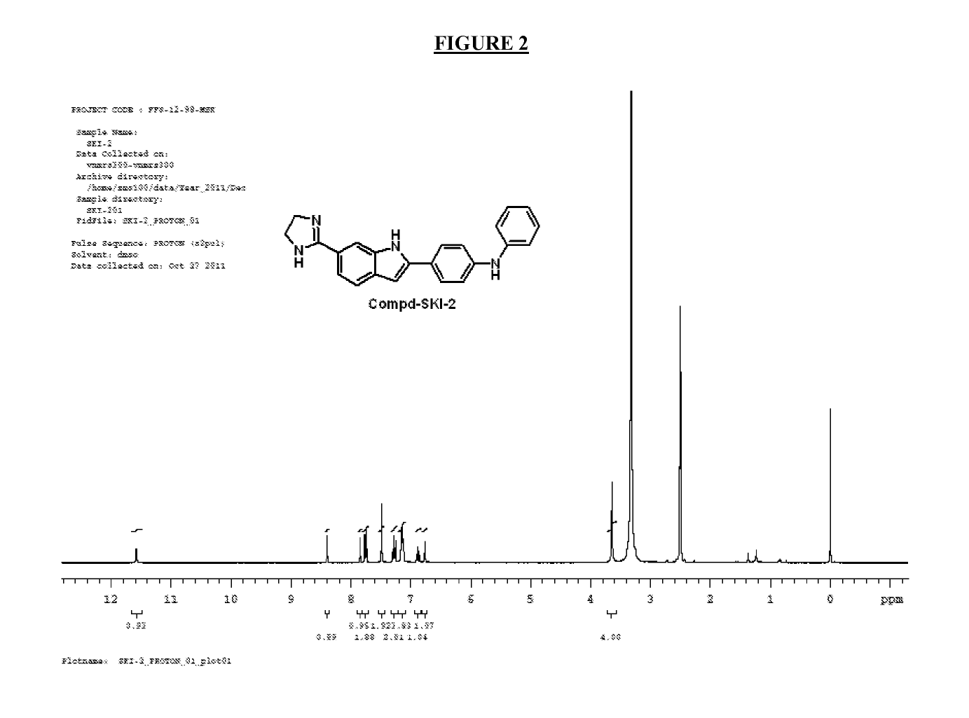

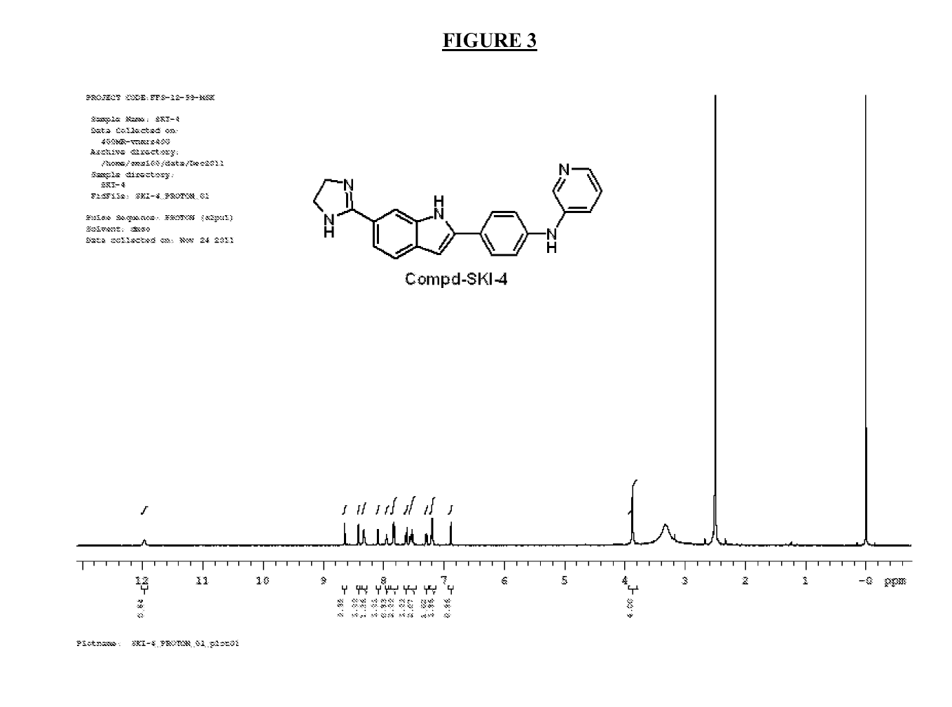

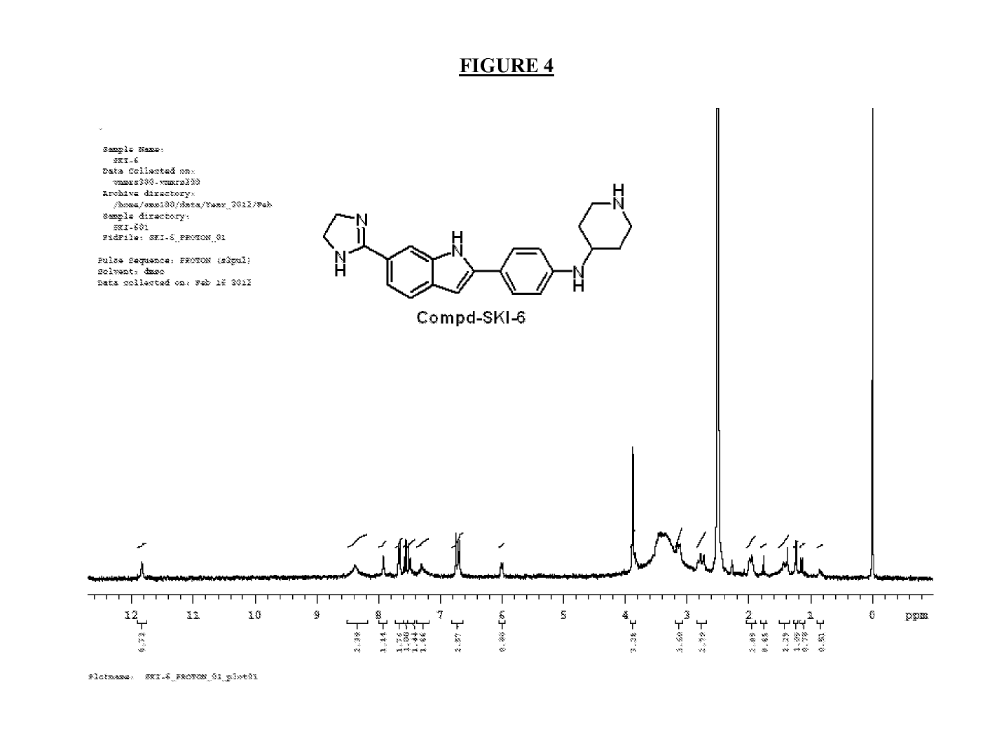

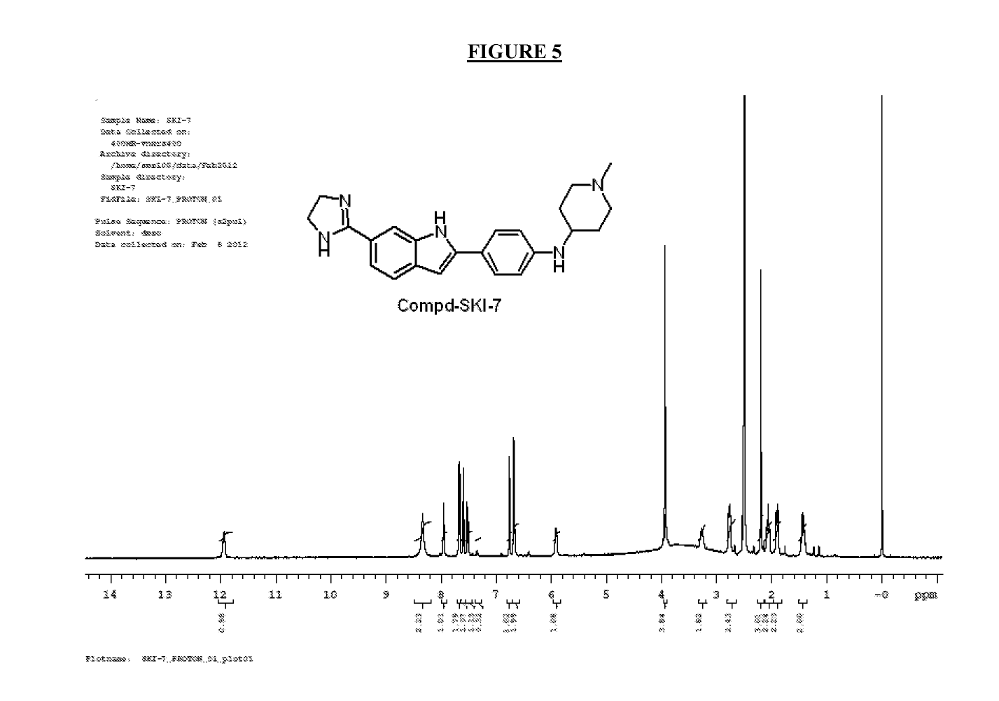

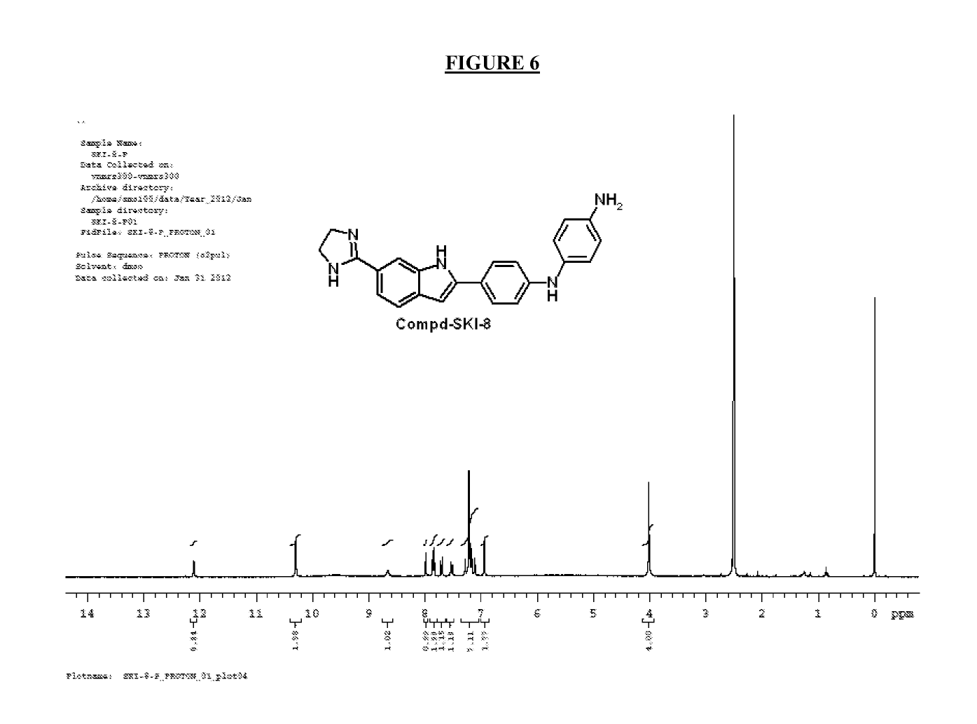

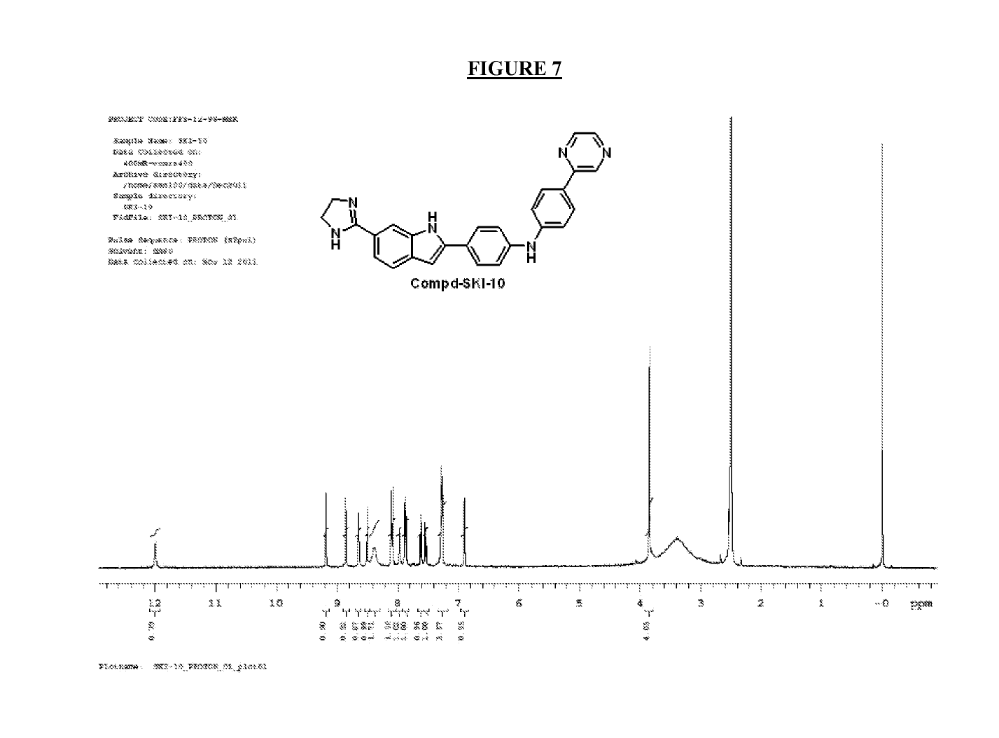

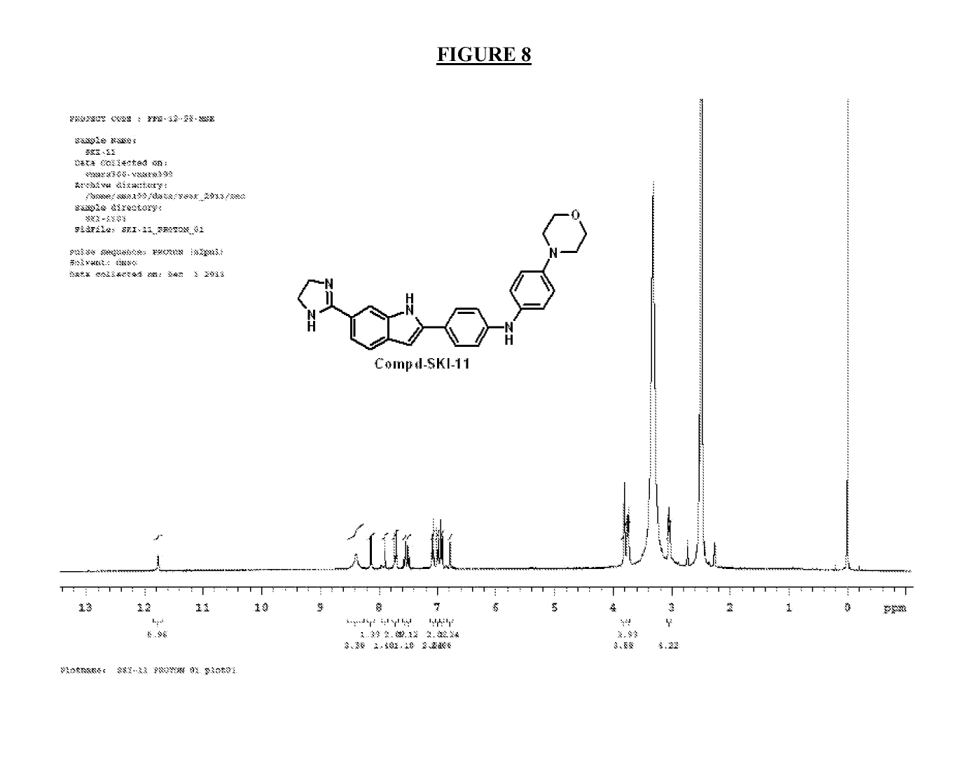

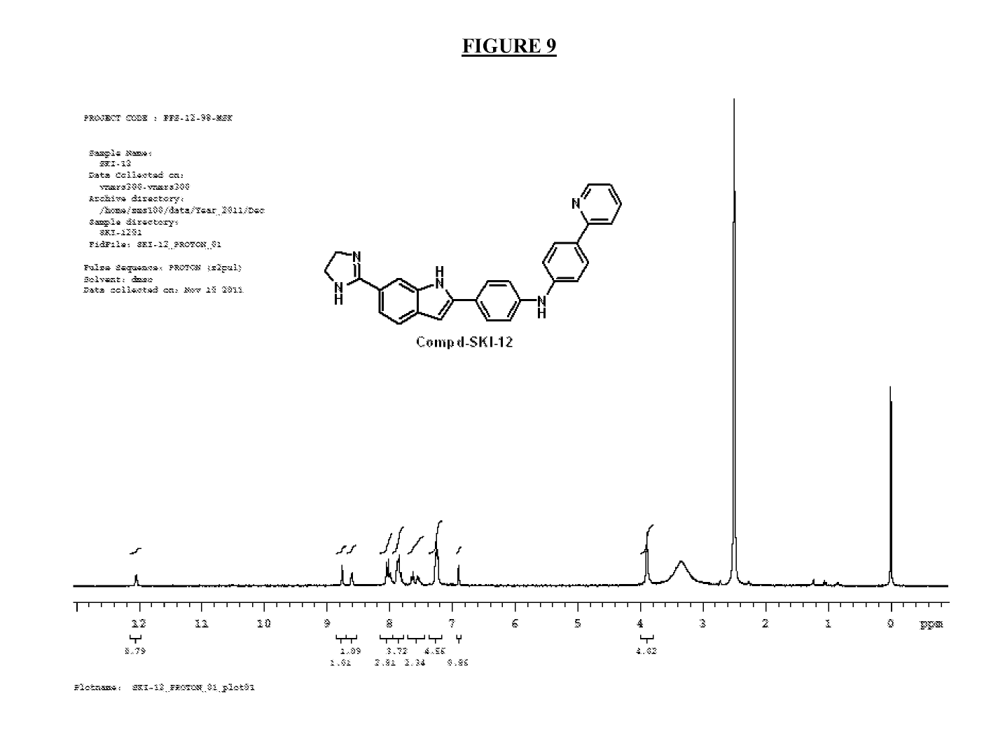

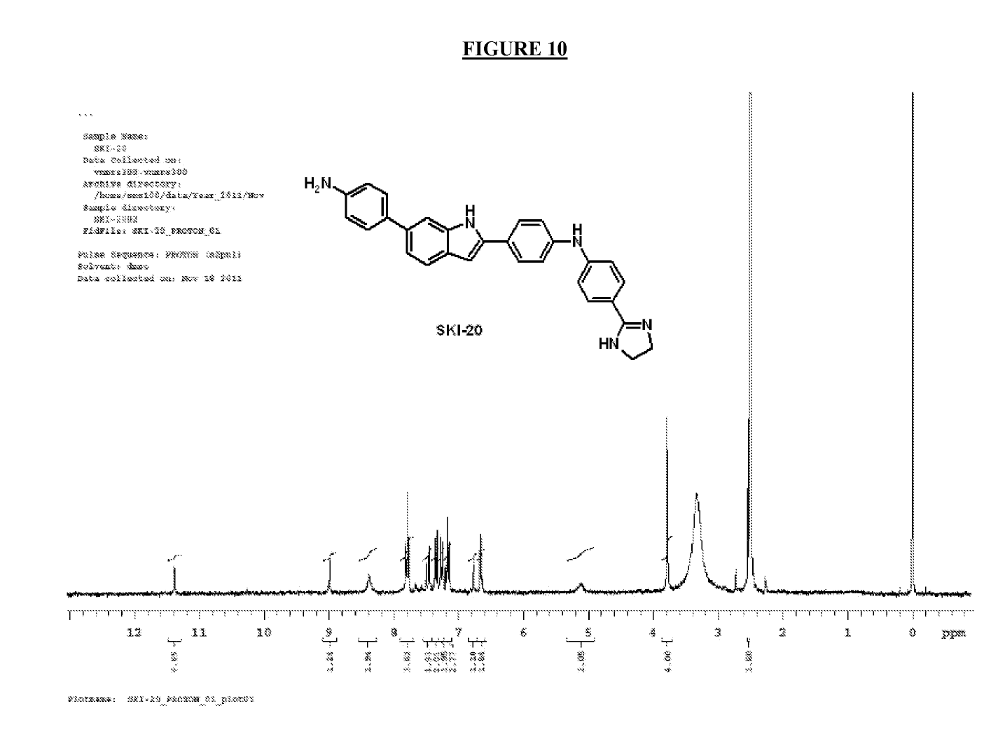

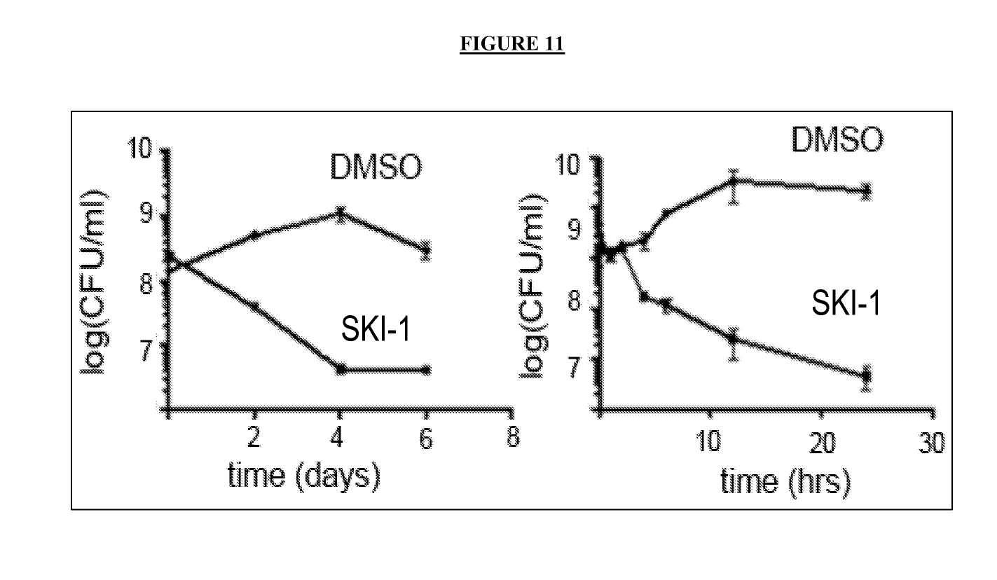

Either Mycobacterium tuberculosis or Mycobacterium smegmatis liquid cultures were incubated with SKI-1 (356313) at a concentration of 1.9 micromolar and viable bacterial titer was determined over the time period indicated by culturing serial dilutions on agar media. Control cultures were treated with an equivalent volume of DMSO. FIG. 1 shows the results of the comparison of control (DMSO) treatment and SKI-1 (356313) treatment in two mycobacteria: Tuberculosis is shown in the left-hand graph, while Mycobacterium smegmatis is shown in the right-hand graph. The graph indicates that incubation with SKI-1 results in a decline in viable bacteria over time, indicating that the drug is killing (i.e. “bactericidal”) rather than just inhibiting growth (i.e. “static”).

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here. The starting materials are either commercially available, synthesized as described in the examples or may be obtained by the methods well known to persons of skill in the art.

Certain compounds of the invention may be synthesized via the following general routes:

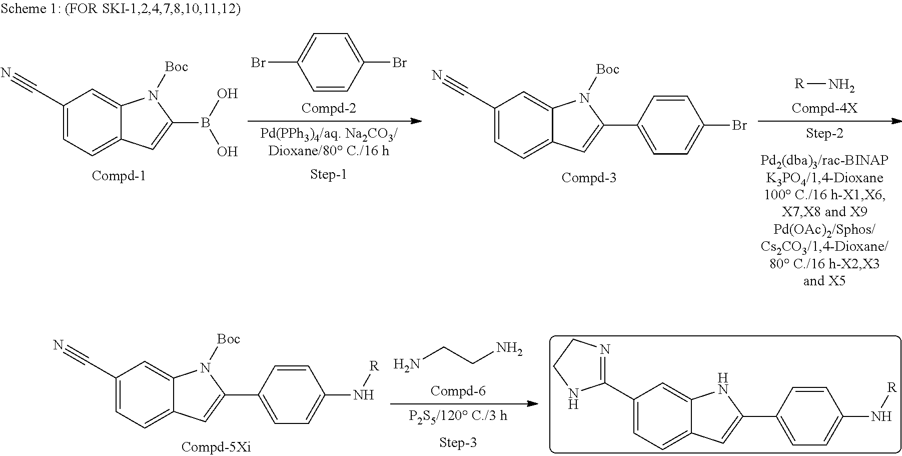

Monomers:

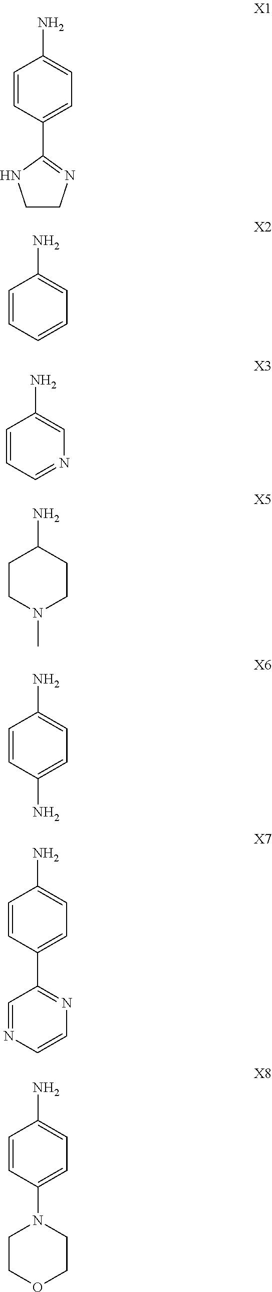

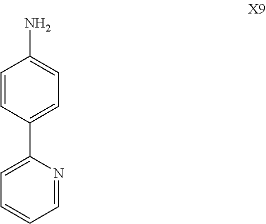 [SMILES: Nc1ccc(-c2ccccn2)cc1]

Detailed descriptions of the synthesis of representative compounds of the invention follow.

Reaction Step-1: To a solution of 1.2 g Compd-1 in 12 mL 1,4-Dioxane was added 1.0 g 1,4-Dibromobenzene, 900 mg Na₂CO₃ and 2 mL water at RT and degassed for 10 mins. Added 50 mg Pd(PPh₃)₄ and stirred at 80° C. for overnight. Solvent was removed under reduced pressure; the residue was dissolved in Ethyl acetate (15 mL) and washed with water (15 mL). Organic layer was dried over anhydrous Sodium sulphate, filtered and concentrated under reduced pressure to give crude Compd-3. The crude compound was purified by column chromatography. Desired product eluted in 4% Ethyl acetate in Petroleum (Pet.) ether. An off-white solid [700 mg (42%)] was obtained. Rf value was 0.6 in 30% Ethyl acetate in pet ether.

Reaction Step-2 (for X1, X6, X7, X8 and X9): To a solution of Compd-3 (150 mg) in 1,4-dioxane (3.0 mL) was added compound-4X (0.453 mmol), K₃PO₄ (240 mg) and rac.BINAP (36 mg) at rt. The resulting reaction mixture was degassed using N₂ for 10 mins. Pd₂(dba)₃ (34.5 mg) was added and stirred at 100° C. for overnight. Reaction progress was monitored by LCMS analysis. The reaction mixture was concentrated under reduced pressure. Diluted reaction mixture with water (10 mL), extracted with DCM (2×10 mL). The combined organic layer was dried over anhydrous sodium sulphate, filtered and filtrate was concentrated under reduced pressure to give the crude compounds of 5Xi. The crude compound was preceded for next step, without any further purification. TLC system: 20% Ethyl acetate in pet ether. Nature/Yield of the compound: Please refer to Table 12. A similar procedure was followed for X1, X6, X7, X8 and X9.

Reaction Step-2 (for X2, X3 and X5): To a solution of Compound-3 (100 mg) in 1, 4-Dioxane (3.0 mL) was added Compound-4X (0.3 mmol), Cs₂CO₃ (244.5 mg) and S-Phos (10 mg). The resulting reaction mixture was degassed using N₂ for 10 mins. Added Pd(OAC)₂ (3 mg) and stirred at 80° C. for overnight. Reaction progress was monitored by LCMS analysis. The reaction mixture was concentrated under reduced pressure. Diluted reaction mixture with water (10 mL), extracted with DCM (2×10 mL). The combined organic layer was dried over anhydrous sodiumsulphate, filtered and filtrate was concentrated under reduced pressure to give the crude compounds of 5Xi. The crude compound was preceded for next step, without any further purification. TLC system: 30% Ethyl acetate in pet ether. Nature/Yield of the compound: Please refer to Table 12. A similar procedure was followed for X2, X3 and X5.

TABLE 12

| | Structure of | M. Wt of | Qty of | Structure of the | | |
|---|---|---|---|---|---|---|
| S. No | monomer | monomer | monomer | product | Yield | Result |
| 1 | 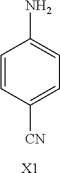 | 118.14 | 83.5 mg | 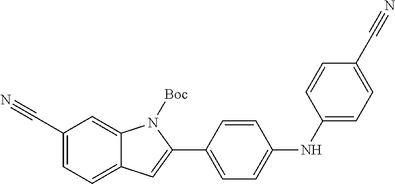 [SMILES: CC(C)(C)OC(=O)n1c(-c2ccc(Nc3ccc(C#N)cc3)cc2)cc2ccc(C#N)cc21] | 180 mg crude | Crude compound proceeded for next step |
| 2 | 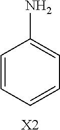 | 93.13 | 27.9 mg | 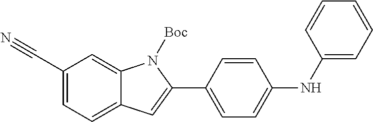 [SMILES: CC(C)(C)OC(=O)n1c(-c2ccc(Nc3ccccc3)cc2)cc2ccc(C#N)cc21] | 202 mg crude | Crude compound proceeded for next step |
| 3 | 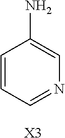 | 94.11 | 42.5 mg | 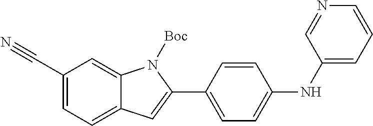 [SMILES: CC(C)(C)OC(=O)n1c(-c2ccc(Nc3cccnc3)cc2)cc2ccc(C#N)cc21] | 156 mg crude | Crude compound proceeded for next step |
| 4 | 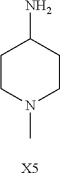 | 114.19 | 64.5 mg | 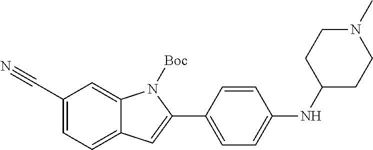 [SMILES: CN1CCC(Nc2ccc(-c3cc4ccc(C#N)cc4n3C(=O)OC(C)(C)C)cc2)CC1] | 182 mg crude | Crude compound proceeded for next step |
| 5 | 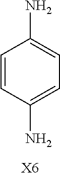 | 108.14 | 117.8 mg | 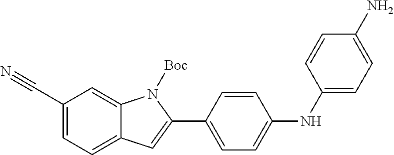 [SMILES: CC(C)(C)OC(=O)n1c(-c2ccc(Nc3ccc(N)cc3)cc2)cc2ccc(C#N)cc21] | 180 mg crude | Crude compound proceeded for next step |
| 6 | 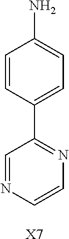 | 171.2 | 96.8 mg | 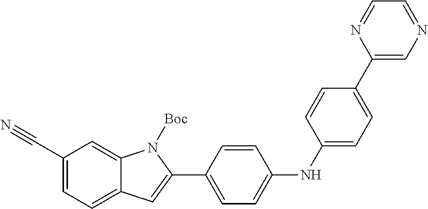 [SMILES: CC(C)(C)OC(=O)n1c(-c2ccc(Nc3ccc(-c4cnccn4)cc3)cc2)cc2ccc(C#N)cc21] | 165 mg crude | Crude compound proceeded for next step |
| 7 | 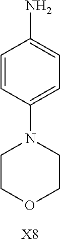 | 178.23 | 100.7 mg | 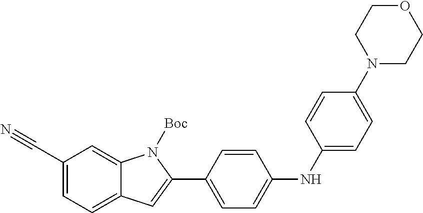 [SMILES: CC(C)(C)OC(=O)n1c(-c2ccc(Nc3ccc(N4CCOCC4)cc3)cc2)cc2ccc(C#N)cc21] | 172 mg | Crude compound proceeded for next step |
| 8 | 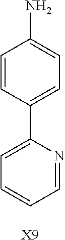 | 170.21 | 96.2 mg | 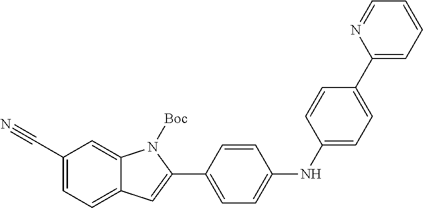 [SMILES: CC(C)(C)OC(=O)n1c(-c2ccc(Nc3ccc(-c4ccccn4)cc3)cc2)cc2ccc(C#N)cc21] | 175 mg crude | Crude compound proceeded for next step |

Reaction Step-3: To Compound-5Xi (100 mg) was added ethane-1,2-diamine (3 mL), P₂S₅ (30 mg) and heated at 120° C. for 3 hrs. The reaction mixture temperature was allowed to RT, poured into ice cold water (15 mL) and obtained solid compound. The crude compound was purified by prep HPLC to provide an off white solid. TLC system: 30% Ethyl acetate in pet ether. Nature/Yield of the compound: Please refer to Table 13.

TABLE 13

| | | M. Wt | Qty of |
|---|---|---|---|
| SN | Structure of 5Xi | of 5Xi | 5Xi |
| 1 | 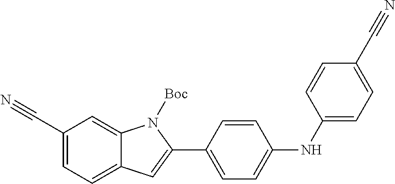 [SMILES: CC(C)(C)OC(=O)n1c(-c2ccc(Nc3ccc(C#N)cc3)cc2)cc2ccc(C#N)cc21] | 434.49 | 80 mg |
| 2 | 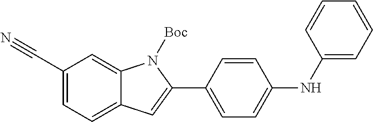 [SMILES: CC(C)(C)OC(=O)n1c(-c2ccc(Nc3ccccc3)cc2)cc2ccc(C#N)cc21] | 409.48 | 110 mg |
| 3 | 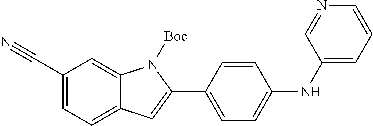 [SMILES: CC(C)(C)OC(=O)n1c(-c2ccc(Nc3cccnc3)cc2)cc2ccc(C#N)cc21] | 410.47 | 41 mg |
| 4 | 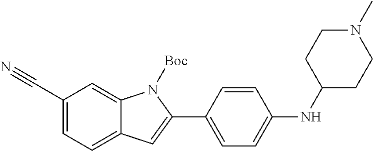 [SMILES: CN1CCC(Nc2ccc(-c3cc4ccc(C#N)cc4n3C(=O)OC(C)(C)C)cc2)CC1] | 430.54 | 120 mg |
| 5 | 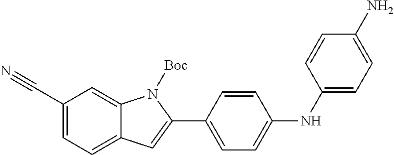 [SMILES: CC(C)(C)OC(=O)n1c(-c2ccc(Nc3ccc(N)cc3)cc2)cc2ccc(C#N)cc21] | 424.49 | 161 mg |
| 6 | 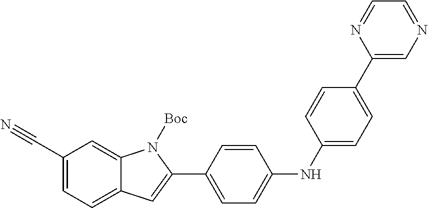 [SMILES: CC(C)(C)OC(=O)n1c(-c2ccc(Nc3ccc(-c4cnccn4)cc3)cc2)cc2ccc(C#N)cc21] | 487.55 | 100 mg |
| 7 | 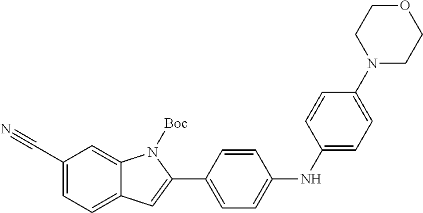 [SMILES: CC(C)(C)OC(=O)n1c(-c2ccc(Nc3ccc(N4CCOCC4)cc3)cc2)cc2ccc(C#N)cc21] | 494.58 | 80 mg |
| 8 | 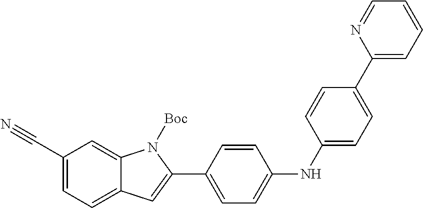 [SMILES: CC(C)(C)OC(=O)n1c(-c2ccc(Nc3ccc(-c4ccccn4)cc3)cc2)cc2ccc(C#N)cc21] | 486.56 | 41 mg |

| | | Qty of | | |
|---|---|---|---|---|
| | Structure and ID of the | Product | | Result & |
| SN | final Target | obtained | Color | Purity |
| 1 | 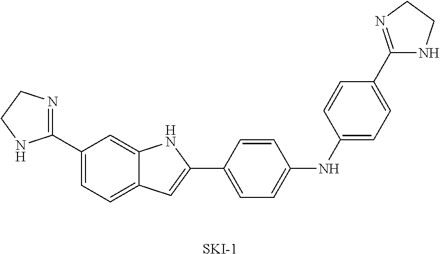 | 15 mg (19%) | Off- white solid | LC-MS: 95.1% HPLC: 97.6% 1H-NMR: OK |
| 2 | 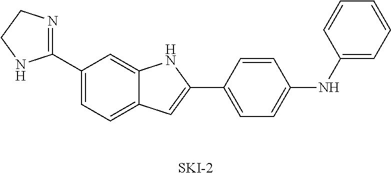 [SMILES: c1ccc(Nc2ccc(-c3cc4ccc(C5=NCCN5)cc4[nH]3)cc2)cc1 ; "SKI-2"] | 78 mg (83%) | Brown solid | LC-MS: 97.9% HPLC: 97.5% 1H-NMR: OK |
| 3 | 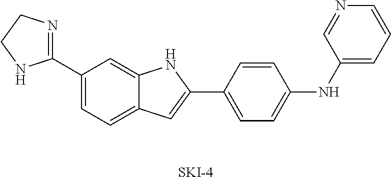 [SMILES: c1cncc(Nc2ccc(-c3cc4ccc(C5=NCCN5)cc4[nH]3)cc2)c1 ; "SKI-4"] | 31 mg (76%) | Off- white solid | LC-MS: 99.4% HPLC: 98.7% 1H-NMR: OK |
| 4 | 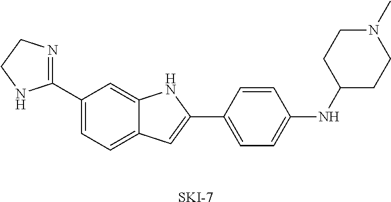 [SMILES: CN1CCC(Nc2ccc(-c3cc4ccc(C5=NCCN5)cc4[nH]3)cc2)CC1 ; "SKI-7"] | 12 mg (12%) | Pale yellow solid | LC-MS: 98.2% HPLC: 99.9% 1H-NMR: OK |
| 5 | 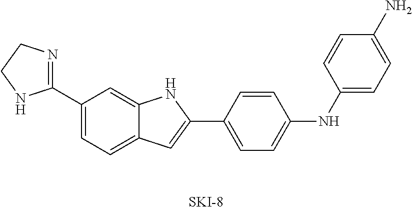 | 40 mg (28%) | Pale yellow solid | LC-MS: 99.7% HPLC: 95.8% 1H-NMR: OK |
| 6 | 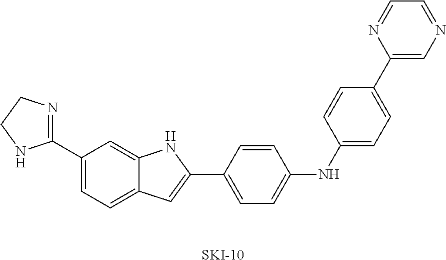 | 20 mg (23%) | Light brown solid | LC-MS: 97.1% HPLC: 98.2% 1H-NMR: OK |
| 7 | 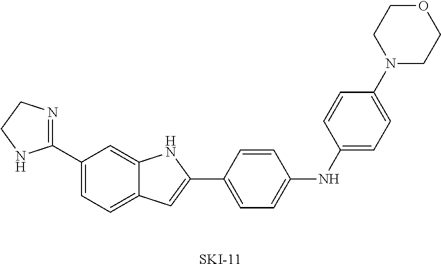 | 7 mg (10%) | Pale yellow solid | LC-MS: 97.4% 1H-NMR: OK |
| 8 | 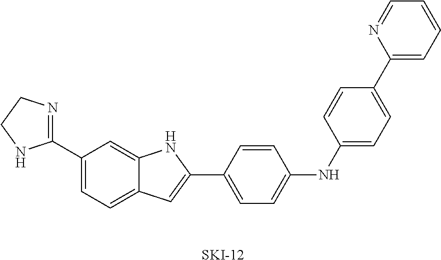 | 30 mg (83%) | Brown solid | LC-MS: 97.4% HPLC: 95.4% 1H-NMR: OK |

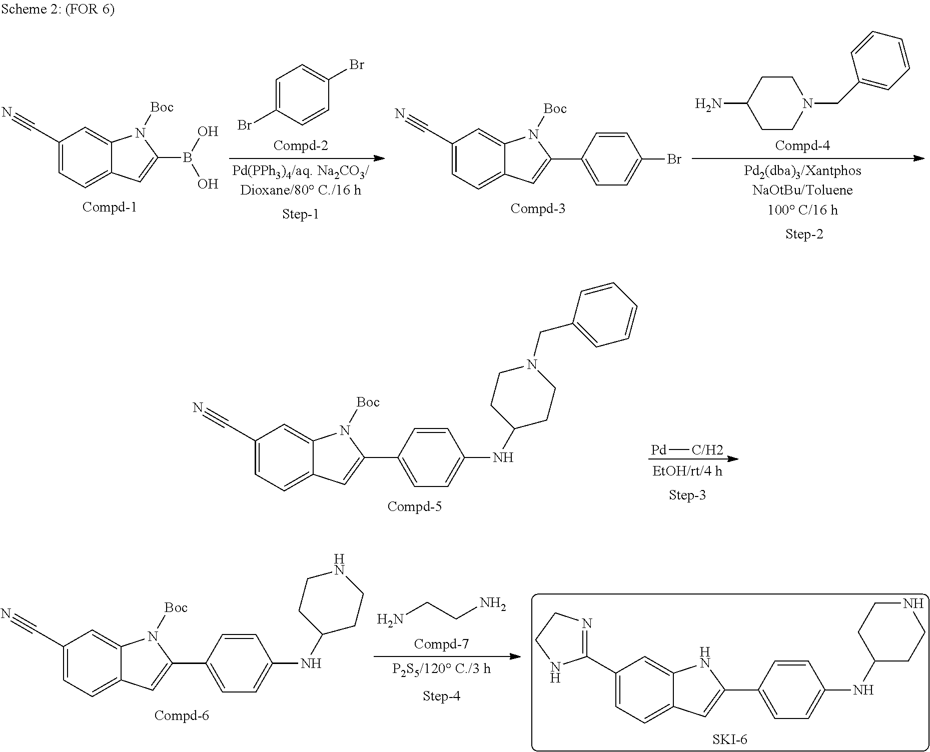

Reaction step-1: To a solution of Compound-1 (1.2 g) in 1,4-Dioxane (12 mL) was added 1,4-Dibromobenzene (1.0 g), Na₂CO₃ (900 mg) and water (2 mL) at RT and degassed for 10 mins. Pd(PPh₃)₄ (50 mg) was added and stirred at 80° C. for overnight. The solvent was removed under reduced pressure; the residue was dissolved in Ethyl acetate (15 mL) and washed with water (15 mL). Organic layer was dried over anhydrous Sodium sulphate, filtered and concentrated under reduced pressure to give crude Compound-3. The crude compound was purified by column chromatography. Desired product eluted in 4% Ethyl acetate in Pet. ether. An off-white solid [700 mg (42%)] was obtained. Rf value was 0.6 in 30% Ethyl acetate in pet ether.

Reaction step-2: To a solution of Compound-3 (50 mg) in toluene (5.0 mL) were added Compound-4 (26 mg), NaOtBu (30 mg) and Xantphos (2.0 mg). The resulting reaction mixture was degassed using N₂ for 10 mins. Pd₂(dba)₃ (2 mg) was added and stirred at 100° C. for overnight. Reaction progress was monitored by LC-MS analysis. The reaction mixture was concentrated under reduced pressure, diluted with water (10 mL) extracted with DCM (2×10 mL). The combined organic layer was dried over anhydrous Na₂SO₄, filtered and concentrated under reduced pressure to give the crude compound. The crude compound was preceded for next step, without any further purification. A brown solid was obtained. Rf value was 0.4 in 30% Ethyl acetate in pet ether.

Reaction step-3: To a solution of Compound-5 (62 mg) in ethanol (6 mL) was added Pd—C (10%, 30 mg) at rt. Resulting reaction mixture was stirred under H₂ (balloon pressure) for 4 hrs at rt. The reaction mixture was filtered through celite, concentrated under reduced pressure. Crude compound was preceded for next step without further purification. A yellow solid was obtained. Rf value was 0.4 in 30% Ethyl acetate in pet ether.

Reaction step-4: To a solution of Compound-6 (45 mg) in ethane 1,2-diamine (3 mL) was added P₂S₅ (12 mg) and heated at 120° C. for 3 hrs. The reaction mixture temperature was allowed to RT, poured into ice cold water (15 mL) and obtained solid compound. The crude compound was purified by prep HPLC. A yellow solid was obtained [2 mg (19%)].

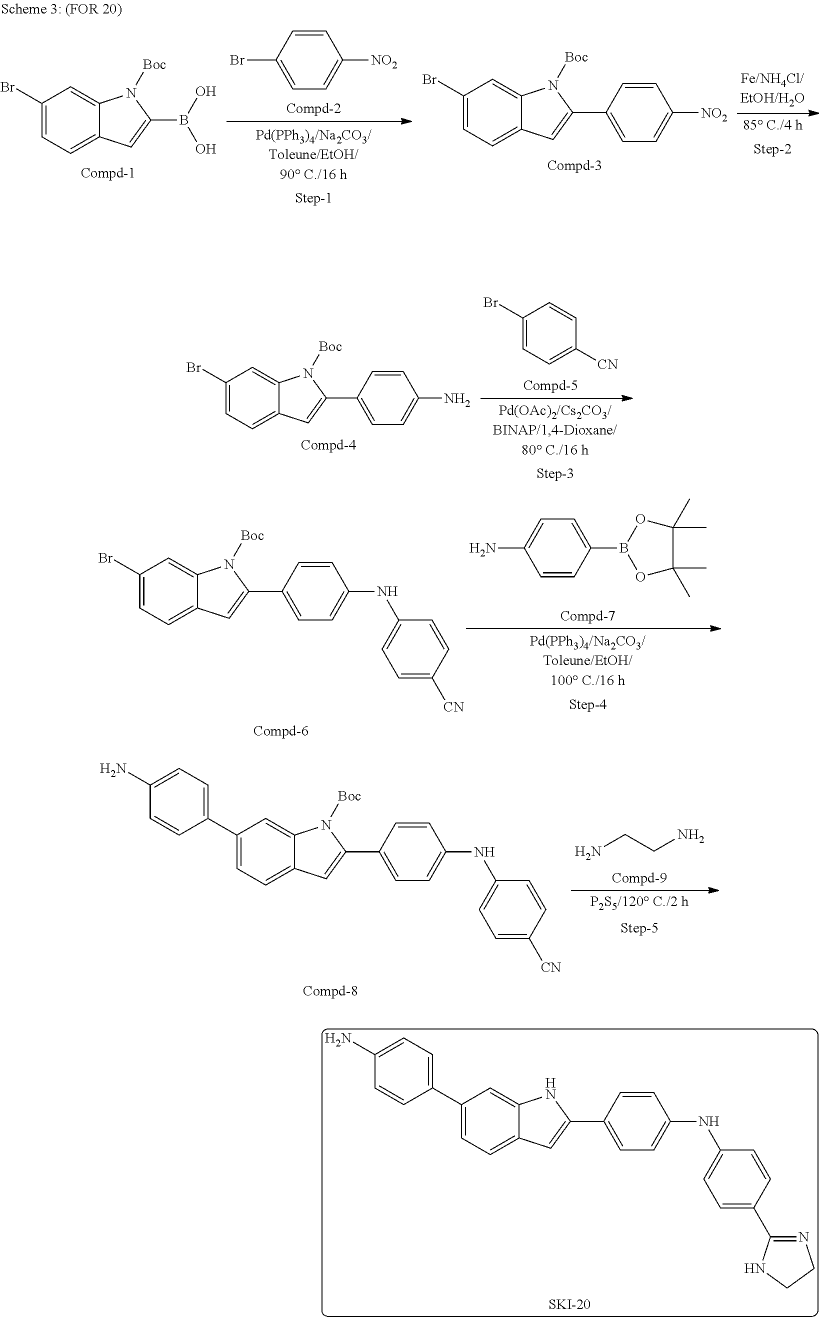

Reaction step-1: To a solution of 1-Bromo-4-nitrobenzene (654 mg) in toluene/ethanol (9:1, 10 mL) was added sodium carbonate (623 mg) followed by Compd-1 (1.0 g) at RT. The reaction mixture was degassed for 15 minutes with argon and Pd(PPh₃)₄ (68 mg) was added at RT. Again degassed for another 5 minutes and the reaction mixture was stirred at 90° C. for 16 hrs under argon. The reaction mixture was concentrated under reduced pressure, obtained crude was dissolved in Ethyl acetate (15 mL) and washed with water (10 mL). Organic layer was dried over anhydrous Sodium sulphate filtered; filtrate was concentrated under reduced pressure to give crude. The crude compound was purified by flash chromatography. The desired product was eluted in 2% ethyl acetate in Pet. ether as pale yellow solid [650 mg (53%)]. Rf value was 0.6 in 10% Ethyl acetate in pet ether.

Reaction step-2: To a solution of Compd-3 (400 mg) in ethanol/water (8:2, 10 mL) was added Iron powder (270 mg) and ammonium chloride (26 mg) at rt. The resulting reaction mixture was stirred at 85° C. for 3-4 hrs. Reaction mixture temperature was allowed to rt, filtered through celite. Ethanol was concentrated under reduced pressure; obtained crude was dissolved in ethyl acetate (10 ml) and washed with water (10 ml). Organic layer was dried over anhydrous sodium sulphate, filtered and concentrated under reduced pressure to give crude. Crude compound was preceded for next step without any further purification. An off-white solid (350 mg crude) was obtained. Rf value was 0.4 in 30% Ethyl acetate in pet ether.

Reaction step-3: To a solution of Compd-4 (300 mg) in 1,4-dioxane (6 mL) was added 4-Bromo benzonitrile (154.9 mg), Palladium acetate (5.2 mg), Cs₂CO₃ (304.6 mg), BINAP (19.2 mg) at RT. Resulting reaction mixture was heated at 80° C. for 16 h. Reaction progress was monitored by TLC. The reaction mixture was concentrated under reduced pressure; the residue was diluted with water (10 mL) and extracted with Ethyl acetate (2×10 mL). Combined organic layer was dried over anhydrous Sodium sulphate and filtered. Filtrate was concentrated under reduced pressure to give the crude Compd-6. The crude compound was purified by column chromatography (100-200 mesh silica gel, 11% EtOAc in Pet-ether as eluent) to get Compd-6. An off-white solid [250 mg (66.1%)] was obtained. Rf value was 0.5 in 15% Ethyl acetate in pet ether.

Reaction step-4: To a solution of Compd-6 (100 mg) in Toluene: water (8:2, 4 mL) was added 4-aminophenylboronate ester (44 mg) and sodium carbonate (43.2 mg) at RT. Reaction mixture was degassed with argon for 10 min. and Pd(PPh₃)₄ (7 mg) was added. The reaction mixture was stirred at 100° C. for 16 h. Reaction progress was monitored by TLC. The reaction mixture was concentrated under reduced pressure; residue was diluted with water (10 mL) and extracted with Ethyl acetate (2×10 mL). Organic layer was dried over anhydrous Sodium sulphate and filtered. Filtrate was concentrated under reduced pressure to give crude compound. The crude compound was purified by column chromatography (100-200 mesh silica gel, 30% EtOAc in Pet-ether as eluent). Compound-8, a yellow gummy solid [35 mg (34.1%)], was obtained. Rf value was 0.3 in 30% Ethyl acetate in pet ether.

Reaction step-5: To a solution of Compound-8 (35 mg) in ethane-1,2-diamine (1.5 mL) was added P₂S₅ (7.7 mg) at RT. Reaction mixture was stirred at 120° C. for 2 h. Reaction progress was monitored by LCMS. Reaction mixture was concentrated under reduced pressure. The crude compound was purified by prep HPLC. A pale yellow solid [10 mg (33.3%)], was obtained.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

1. A compound according to formula (I) 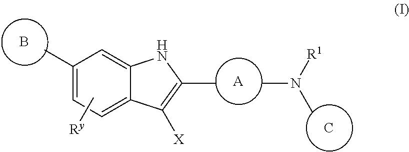
wherein
R¹ is selected from hydrogen and C₁₋₆ alkyl;
Ring A is selected from phenyl, thiophene and furan, wherein said phenyl, thiophene or furan may be optionally substituted with 1, 2, 3 or 4 R^{a} groups;
R^{a} is selected in each instance from hydrogen, halogen, C₁₋₆ alkyl, C₁₋₆ haloalkyl, C₁₋₆ alkoxy, C₁₋₆ haloalkoxy, amino, cyano, and nitro;
Ring B is selected from
a) phenyl, thiophene and furan, substituted with at least one nitrogen-containing moiety, and further optionally substituted with one or more C₁₋₆ alkyl and/or C₁₋₆ alkoxy groups; and
b) a nitrogen-containing heterocyclyl, wherein said heterocyclyl may be optionally substituted with 1, 2, 3, 4 or 5 R^{b} groups;
R^{b} is selected in each instance from hydrogen, halogen, C₁₋₆ alkyl, C₁₋₆ haloalkyl, cyano, and R^{d};
R^{d} is chosen from carbocyclyl and heterocyclyl, wherein said carbocyclyl or heterocyclyl may be optionally substituted with 1, 2, 3, 4 or 5 substituents selected from halogen, C₁₋₆ alkyl, C₁₋₆ haloalkyl, C₁₋₆ alkoxy, C₁₋₆ haloalkoxy, nitro, amino, and cyano;
with the proviso that no more than one R^{b} may be R^{d};
Ring C is selected from heterocyclyl and carbocyclyl, wherein Ring C may be optionally substituted with 1, 2, 3, 4 or 5 R^{c} groups;
R^{c} is selected in each instance from hydrogen, halogen, C₁₋₆ alkyl, C₁₋₆ haloalkyl, C₁₋₆ alkoxy, C₁₋₆ haloalkoxy, cyano, nitro, amino, and R^{e};
R^{e} is chosen from carbocyclyl and heterocyclyl, wherein said carbocyclyl or heterocyclyl may be optionally substituted with 1, 2, 3, 4 or 5 substituents selected from halogen, C₁₋₆ alkyl, C₁₋₆ haloalkyl, C₁₋₆ alkoxy, C₁₋₆ haloalkoxy, cyano, nitro and amino;
with the proviso that no more than one R^{c} may be R^{e};
R^{y} represents one, two or three groups individually selected from hydrogen, halogen, C₁₋₆ alkyl, C₁₋₆ haloalkyl, C₁₋₆ alkoxy, C₁₋₆ haloalkoxy, amino, cyano and nitro; and
X is selected from hydrogen, halogen, C₁₋₆ alkyl and C₁₋₆ haloalkyl;
with the proviso that when Ring B is imidazoline and Ring A is phenyl, Ring C is not triazine; and
with the proviso that the compound is not 6-(4,5-dihydro-1H-imidazol-2-yl)-2-[4-[[4-(4,5-dihydro-1H-imidazol-2-yl)phenyl]amino]phenyl]-1H-indole.

2. A compound according to claim 1 wherein Ring A is phenyl.

3. A compound according to claim 2 wherein Ring A is unsubstituted phenyl.

4. A compound according to claim 1 wherein R¹ is hydrogen or methyl.

5. (canceled)

6. A compound according to claim 1 wherein Ring B is unsubstituted imidazoline.

7. A compound according to claim 1 wherein Ring B is phenyl substituted with amino and/or a nitrogen-containing monocycle.

8. A compound according to claim 7 wherein Ring B is phenyl and substituted with amino, morpholinyl and/or pyridinyl.

9. A compound according to claim 1 wherein Ring C is phenyl and R^{c} is selected from hydrogen, a nitrogen-containing monocycle, amino and nitro.

10. (canceled)

11. A compound according to claim 1 wherein Ring C is a nitrogen-containing monocycle and R^{c} is hydrogen or C₁₋₆ alkyl.

12. A compound according to claim 1 wherein R^{y} is hydrogen, halogen, methyl or trifluoromethyl.

13. A compound according to claim 12 wherein R^{y} is hydrogen.

14. A compound according to claim 1 wherein X is hydrogen, halogen, methyl or trifluoromethyl.

15. A compound according to claim 14 wherein X is hydrogen.

16. A compound according to claim 1 wherein
R¹ is hydrogen or methyl;
Ring A is optionally substituted phenyl;
Ring B is selected from optionally substituted imidazoline and phenyl substituted with amino and/or a nitrogen-containing monocycle;
R^{y} is hydrogen, halogen, methyl or trifluoromethyl;
X is hydrogen, halogen, methyl or trifluoromethyl; and
Ring C is selected from:
phenyl, and R^{c} is selected from hydrogen, a nitrogen-containing monocycle, amino and nitro; and
a nitrogen-containing monocycle, and R^{c} is hydrogen or C₁₋₆ alkyl.

17. (canceled)

18. A compound according to claim 1 of the formula

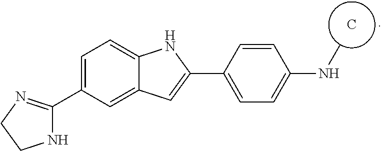

19. A compound according to claim 18 wherein R^{c} is para-substituted.

20. A pharmaceutical composition comprising a compound according to claim 1 and a pharmaceutically acceptable carrier.

21. A method of treating a bacterial or fungal infection in a subject in need thereof, comprising administering to the subject a therapeutically effective amount of a compound according to claim 1 or a compound of formula (II) 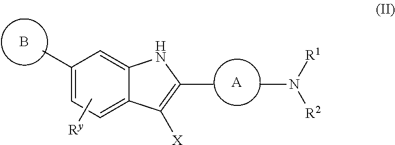
wherein
R¹ is selected from hydrogen and C₁₋₆ alkyl;
R² is hydrogen, C₁₋₆ alkyl, or a ring selected from heterocyclyl and carbocyclyl, wherein said ring may be optionally substituted with 1, 2, 3, 4 or 5 R groups;
R is selected in each instance from hydrogen, halogen, C₁₋₆ alkyl, C₁₋₆ haloalkyl, cyano, nitro, amino, carbocyclyl and heterocyclyl, wherein only one instance of R is carbocyclyl or heterocyclyl;
Ring A is selected from phenyl, thiophene and furan, wherein said phenyl, thiophene or furan may be optionally substituted with 1, 2, 3 or 4 R^{a} groups;
R^{a} is selected in each instance from hydrogen, halogen, C₁₋₆ alkyl, C₁₋₆ haloalkyl, C₁₋₆ alkoxy, C₁₋₆ haloalkoxy, amino, cyano, and nitro;
Ring B is selected from
a) a) phenyl, thiophene and furan, substituted with at least one nitrogen-containing moiety, and further optionally substituted with one or more C₁₋₆ alkyl and/or C₁₋₆ alkoxy groups; and
b) a nitrogen-containing heterocyclyl, wherein said heterocyclyl may be optionally substituted with 1, 2, 3, 4 or 5 R^{b} groups;
R^{b} is selected in each instance from hydrogen, halogen, C₁₋₆ alkyl, C₁₋₆ haloalkyl, cyano, and R^{d};
R^{d} is chosen from carbocyclyl and heterocyclyl, wherein said carbocyclyl or heterocyclyl may be optionally substituted with 1, 2, 3, 4 or 5 substituents selected from halogen, C₁₋₆ alkyl, C₁₋₆ haloalkyl, C₁₋₆ alkoxy, C₁₋₆ haloalkoxy, nitro, amino, and cyano;
with the proviso that no more than one R^{b} may be R^{d};
R^{y} represents one, two or three groups individually selected from hydrogen, halogen, C₁₋₆ alkyl, C₁₋₆ haloalkyl, C₁₋₆ alkoxy, C₁₋₆ haloalkoxy, cyano and nitro; and
X is selected from hydrogen, halogen, C₁₋₆ alkyl and C₁₋₆ haloalkyl;
with the proviso that when the compound is 6-(4,5-dihydro-1H-imidazol-2-yl)-2-[4-[[4-(4,5-dihydro-1H-imidazol-2-yl)phenyl]amino]phenyl]-1H-indole, the bacteria is not Bacillus anthracis.

22-26. (canceled)

27. A method according to claim 21, wherein the compound is selected from

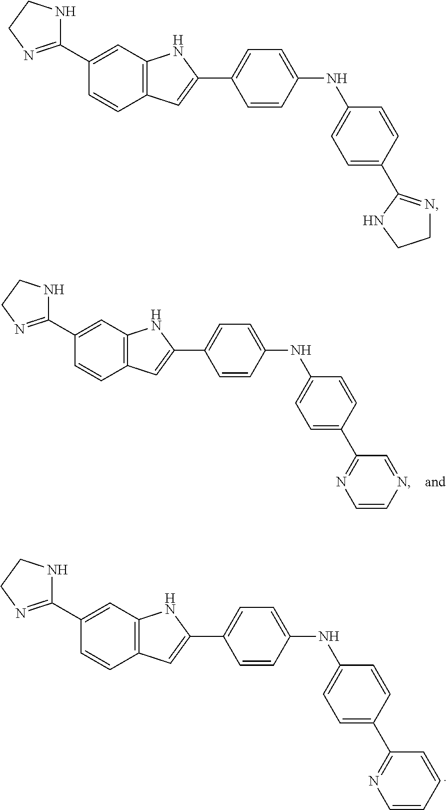 [SMILES: c1cc(C2=NCCN2)ccc1Nc1ccc(-c2cc3ccc(C4=NCCN4)cc3[nH]2)cc1 ; c1cnc(-c2ccc(Nc3ccc(-c4cc5ccc(C6=NCCN6)cc5[nH]4)cc3)cc2)cn1 ; "and" ; c1ccc(-c2ccc(Nc3ccc(-c4cc5ccc(C6=NCCN6)cc5[nH]4)cc3)cc2)nc1]

28. (canceled)

29. A method of killing or inhibiting the growth of bacteria or fungus, comprising contacting the bacteria or fungus with a compound according to claim 1 or a compound of formula (II) 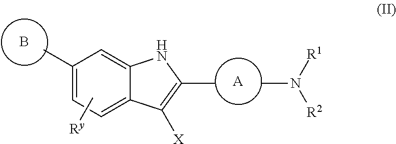
wherein
R¹ is selected from hydrogen and C₁₋₆ alkyl;
R² is hydrogen, C₁₋₆ alkyl, or a ring selected from heterocyclyl and carbocyclyl, wherein said ring may be optionally substituted with 1, 2, 3, 4 or 5 R groups;
R is selected in each instance from hydrogen, halogen, C₁₋₆ alkyl, C₁₋₆ haloalkyl, cyano, nitro, amino, carbocyclyl and heterocyclyl, wherein only one instance of R is carbocyclyl or heterocyclyl;
Ring A is selected from phenyl, thiophene and furan, wherein said phenyl, thiophene or furan may be optionally substituted with 1, 2, 3 or 4 R^{a} groups;
R^{a} is selected in each instance from hydrogen, halogen, C₁₋₆ alkyl, C₁₋₆ haloalkyl, C₁₋₆ alkoxy, C₁₋₆ haloalkoxy, amino, cyano, and nitro;
Ring B is selected from
a) phenyl, thiophene and furan, substituted with at least one nitrogen-containing moiety, and further optionally substituted with one or more C₁₋₆ alkyl and/or C₁₋₆ alkoxy groups; and
b) a nitrogen-containing heterocyclyl, wherein said heterocyclyl may be optionally substituted with 1, 2, 3, 4 or 5 R^{b} groups;
R^{b} is selected in each instance from hydrogen, halogen, C₁₋₆ alkyl, C₁₋₆ haloalkyl, cyano, and R^{d};
R^{d} is chosen from carbocyclyl and heterocyclyl, wherein said carbocyclyl or heterocyclyl may be optionally substituted with 1, 2, 3, 4 or 5 substituents selected from halogen, C₁₋₆ alkyl, C₁₋₆ haloalkyl, C₁₋₆ alkoxy, C₁₋₆ haloalkoxy, nitro, amino, and cyano;
with the proviso that no more than one R^{b} may be R^{d};
R^{y} represents one, two or three groups individually selected from hydrogen, halogen, C₁₋₆ alkyl, C₁₋₆ haloalkyl, C₁₋₆ alkoxy, C₁₋₆ haloalkoxy, amino, cyano and nitro; and
X is selected from hydrogen, halogen, C₁₋₆ alkyl and C₁₋₆ haloalkyl.

30-44. (canceled)
